SEQUENCE LISTING (4 sequences)

SEQ ID NO: 1 - protein, 7 aa, synthetic construct
CVRARTR

SEQ ID NO: 2 - RNA, 786 nt, Homo sapiens
  source 1..786: mol_type = other RNA
  unsure 1..786: note = long non-coding RNA
gaaggtagaaggtggggtctgccggacgcgtgttcctgccaccaggtgcccgctccccgcgaggccggctcaggagcagaaggaagcccggtgccagccagccttcctgaaagaccaagcccgcgccatccggcttcctccagtggacgcctgcaggacccaggaatgtttttcttgaaggcatccagcatctccagttagcagtactgattttttttccccccaacaaaggaacactacatcaacactgttggcggggacctggacacagaagactcctgtttcaagaaaatacaatcatctctcaaaggctgtaatttatatgcattttaaaactctaggcattgaaaaccacccaagtgtcccaaatagaagggtaatatataatcaatcactcagtgtaatattatacatcctttaaaaatgttattgggaaatgttttatgatctgtaagtccaaggaatcctctcccaccatttctttccccccgctgttcccccatacccacacttctttgttccaattggcatgtaaatttggttttcccgccaaatgagtcagtcatgatgggaacctcaactgatttgaacagatgtgtgtcaatgttacttggaaaactagatgtcaataaccagggtcacagaaaaaggcagtggtcacaactctgtaaaaatgtatgcatgcacacagacaagaactaaagtggaaccccacacaggaaaacagtgggctgtactccagtgctgggacattgaatgactgtatgctgcttttgatttccgtta

SEQ ID NO: 3 - RNA, 949 nt, Homo sapiens
  source 1..949: mol_type = other RNA
  unsure 1..949: note = long non-coding RNA
agctcatataaggtaaaaggcagagtggcctctgtggccagggagccgcaggcaagggactaaggggaggggggctcagtgccagctgcttaaaaatgcccctgtggcagcgaggggcaccagaggctgggtctaattagttgagaagcagtgacacccccaaccactccccaaacaggctggctcccgtctccaggccccaaggagccacacctggaccagaccccaggaaaggcaagggcgctagctggtgggagccaccccgccatgctgatgtcagagaagcaagaactctggagaagcagcctcctgggaccagaggagggccagcagcaggcagcccggagacagaactctcacccacgcctctccaagcctcccaacagaagacagaggtcccccacagccagagacatttcctgaagacatggggaacacagaggcagaaacagcccatccacccaggagctgtcccccacactgccgggagccggcacccagagccgccaggtaaaactgaggccacctggttcaacatcacctttcacagaaggggaagcagccacagaaagaagggcctcgttaagaagtggaacctgggacccccaagcggtgtctctcatcctgactggggatccagagtaggagggagcctttggtggggttggagtcccgccacaggccaccagagcggagcagcgcagcgccctgtctcccagcctgaggtgcagtgctgcatctctggtcagttgggagtctgagatgaagcactgtagctcaggaagagagaagttgttctgcagccatcagcctggaagtgcctggctggtgggccttcttgcagtagcttcccctggagaagaggaaaagcaaaccttcattgagacccaagcggtctctcctgtgctctgtgacaataataaagttccagcccttggcaaaaaaaaaa

SEQ ID NO: 4 - RNA, 1013 nt, Homo sapiens
  source 1..1013: mol_type = other RNA
  unsure 1..1013: note = long non-coding RNA
agctcatataaggtaaaaggcagagtggcctctgtggccagggagccgcaggcaagggactaaggggaggggggctcagtgccagctgcttaaaaatgcccctgtggcagcgaggggcaccagaggctgggtctaattagttgagaagcagtgacacccccaaccactccccaaacaggctggctcccgtctccaggccccaaggagccacacctggaccagaccccaggaaagacatgtggtggaaaccctacggccctggagcccaaggagagcaacggctgtaacaagtgcacaggcaagggcgctagctggtgggagccaccccgccatgctgatgtcagagaagcaagaactctggagaagcagcctcctgggaccagaggagggccagcagcaggcagcccggagacagaactctcacccacgcctctccaagcctcccaacagaagacagaggtcccccacagccagagacatttcctgaagacatggggaacacagaggcagaaacagcccatccacccaggagctgtcccccacactgccgggagccggcacccagagccgccaggtaaaactgaggccacctggttcaacatcacctttcacagaaggggaagcagccacagaaagaagggcctcgttaagaagtggaacctgggacccccaagcggtgtctctcatcctgactggggatccagagtaggagggagcctttggtggggttggagtcccgccacaggccaccagagcggagcagcgcagcgccctgtctcccagcctgaggtgcagtgctgcatctctggtcagttgggagtctgagatgaagcactgtagctcaggaagagagaagttgttctgcagccatcagcctggaagtgcctggctggtgggccttcttgcagtagcttcccctggagaagaggaaaagcaaaccttcattgagacccaagcggtctctcctgtgctctgtgacaataataaagttccagcccttggcaaaaaaaaaa

(19) United States
(12) Patent Application Publication
(10) Pub. No.: US 2025/0145994 A1
(43) Pub. Date: May 8, 2025

(54) COMPOSITIONS AND METHODS FOR TREATING CONDITIONS RELATED TO CELLULAR SENESCENCE

(71) Applicant: UNIVERSITY OF SOUTH FLORIDA, Tampa, FL (US)

(72) Inventors: Wanling XUAN, Tampa, FL (US); Srinivas TIPPARAJU, Tampa, FL (US); Vijaykumar SUTARIYA, Tampa, FL (US)

(21) Appl. No.: 18/941,514

(22) Filed: Nov. 8, 2024

Related U.S. Application Data
(60) Provisional application No. 63/597,238, filed on Nov. 8, 2023.

(51) Int. Cl. C12N 15/113; A61K 47/26; A61K 47/68; A61K 47/69

(52) U.S. Cl. CPC C12N 15/113; A61K 47/26; A61K 47/6891; A61K 47/6901; C12N 2310/141

(57) ABSTRACT

Described herein are extracellular vesicles and methods of using the extracellular vesicles. The extracellular vesicles can include a targeting peptide and an active agent, where the targeting peptide targets senescent cells.

### CROSS-REFERENCE TO RELATED APPLICATION

This application claims priority to, and the benefit of U.S. Provisional Application 63/597,238, filed on Nov. 8, 2023, the contents of which is hereby incorporated in its entirety.

### REFERENCE TO SEQUENCE LISTING

The Sequence Listing submitted Nov. 8, 2024, as a text filed named “11001_204US1_SEQUENCE_LISTING” created Nov. 6, 2024, and having a file size of 8,375 bytes is hereby incorporated by reference pursuant to 37 C.F.R. § 1.52 (e) (5).

### BACKGROUND

Senescent cells accumulate in tissues and organs of individuals as they age and are found at sites of age-related pathologies. Senescent cells are believed important to inhibiting proliferation of dysfunctional or damaged cells and particularly to constraining development of malignancy. The presence of senescent cells in an individual may contribute to aging and aging-related dysfunction. Given that senescent cells have been causally implicated in certain aspects of age-related decline in health and may contribute to certain diseases the presence of senescent cells may have deleterious effects on millions of patients worldwide. Thus, there is a need for methods of targeting senescent cells and methods for treating or preventing conditions, diseases, or disorders related to, associated with, or caused by cellular senescence.

The compositions and methods disclosed herein address these and other needs.

### SUMMARY

Described herein are extracellular vesicles including a targeting peptide and an active agent. In some embodiments, the targeting peptide targets senescent cells. In some embodiments, the targeting peptide can be a programmed death-ligand 1 (PD-L1) binding peptide having at least 80% sequence identity to SEQ ID NO: 1. In some embodiments, the targeting peptide can be conjugated to a PEG-conjugated phospholipid derivative.

Described herein are also pharmaceutical compositions including a pharmaceutically acceptable carrier and an effective amount of the extracellular vesicles described herein.

Also, described herein are methods for delivering an active agent into a senescent cell, including: introducing into the cell an extracellular vesicle described herein or a composition described herein.

In some embodiments, described herein are also methods for preventing cellular senescence, the method including administering to a subject in need thereof an extracellular vesicle described herein or a composition described herein.

Described herein are also methods for restoring expression levels of MIR503HG in senescent VSMCs to reduce cellular senescence, the method including administering to a subject in need thereof an extracellular vesicle including a therapeutically effective amount of a long non-coding RNAs (lncRNAs) coding for a MIR503HG nucleic acid sequence having at least 80% sequence identity to SEQ ID NO: 2, and a targeting peptide, wherein the targeting peptide targets senescent cells.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 shows a scheme of drug delivery and engineering-based.

FIGS. 2A-2C show a scheme for generation of PSC derived contractile VSMCs-Activation of Notch1 signaling promote maturation of iPS cell derived VSMCs (2A). (2B-2C) Specific miRNAs or IncRNA in Extracellular Vesicles (EVs) from PSC derived contractile VSMCs. (2B) is a graph of relative expression of miR-143, miR-145, and miR-503 in EVs-MSCs, EVs-SMCs^{TGF-β}, and EVs-SMCs^{NICD}. (2C) is a graph of relative expression of miR-143HG in EVs-MSCs, EVs-SMCs^{TGF-β}, and EVs-SMCs^{NICD}.

FIG. 3 shows a scheme for generating PSC derived contractile vascular smooth muscle cells (VSMCs) and extracellular vehicles (EVs).

FIGS. 4A-4D (4A) Generation of mesenchymal stem-like cells (MSLCs) neural crest like stem cells (NCLCs) and vascular smooth muscle cells (VSMCs) from human induced pluripotent stem cells (hiPSC). (4B) Differentiated MSLCs express MSLCs markers: CD105 and NG2, with low expression of α-SMA. Calponin expression is undetectable. (4C) Differentiated MSLC/NCLC can further differentiate into osteocytes, chondrocytes and adipocytes as indicated by alizarin red, alcian blue and oil red O staining respectively. (4D) Contractile VSMCs markers calponin and Myh11 by immunostaining in VSMCs induced via TGF-β or Notch1 activation. N1ICD: Notch1 intracellular domain. Bar=100 μm.

FIGS. 5A-5B (5A) Preferred internalization of EVs-VSMCs^{N1ICD} in human aortic smooth muscle cells (HAoSMC) at early passage compared with HAoSMC at late passage or human aortic endothelia cells (HAEC) in a co-culture system with 3 h incubation. EVs were labeled with PKH26 dye. WGA was used for cell boundary visualization and nuclei was stained with DAPI. (5B) Quantitation of EVs internalization (PKH26 intensity normalized with cell numbers). ****P<0.0001, n=8. A.U.: arbitrary unit.

FIGS. 6A-6J (6A) Increased programmed death-ligand 1 (PD-L1) expression in female replicative senscenct HAoSMC (P10) compared with cells from early passage (P2) by immunofluorescent staining. Co-expression of PD-L1 and p16INK4a in HAoSMC are indicated as white arrows. (6B) Enhanced PD-L1 expression in aortic VSMCs from 22M old female mice compared with 3M old young mice. PD-L1 fluorescence intensity was quantitated. n=5. (6C) Chemical structure of the PD-L1 binding peptide (PD-L1-BP): CVRARTR and schematic illustration of the conjugating reaction between DMPE-PEG-Maleimide (DMPE-PEG-MAL) and cysteine-terminated PD-L1-BP. (6D) Modification of the surface of EVs with PD-L1 binding peptide (PD-L1-BP) by hydrophobic insertion using DMPE-PEG for targeting delivery of senescent VSMCs. (6E) The chemical structure of the PD-L1-BP conjugates was characterized by NMR. Top NMR spectrum shows methylene protons adjacent to H-S (3.05 ppm). Bottom NMR spectrum shows absence of methylene protons at 3.05 ppm due to change in chemical environment (in black circle). This confirms sulfide bond conjugation between the cysteine of PD-L1 peptide and maleimide of the DMPE-PEG-MAL. (6F-6H) Enhanced internalization of Cy5.5-PD-L1-BP-EVs (Cy5.5, purple) in female HAoSMC at P10 (WGA, green) compared with Cy5.5-PEG-EVs. The fluorescent dye Cy5.5 was conjugated to the amino group of cysteine to visualize PD-L1-BP. The conjugate of Cy5.5-PEG was used as control. The nuclei were counterstained with DAPI (blue). n=8 from 2 independent experiments. (6I-6J) In vivo delivery of Cy5.5-PD-L1-BP-EVs (50 μg) in aged female mice showed better uptake in aorta compared with Cy5.5-PEG-EVs. n=4 form 4 sections of 2 aged mice in each group. White arrows indicated uptaken EVs. ***P<0.001. A.U.: Arbitrary unit.

FIGS. 7A-7D (7A) Transmission electron microscopy (TEM) images showing extracellular vesicles (EVs) from iPSCs derived MSCLCs and VSMCs differentiated by TGFβ or overexpression with N1ICD. Black arrows indicate EVs with bilayer structure. (7B) Expression of EVs markers (CD9 and CD63) by Western blot. (7C-7D) Higher enrichment of lncRNAs: MIR503HG and MIR143HG (7C); and miRNAs: miR-503, miR-143 and miR-145 (7D) in EVs from VSMCs^{N1ICD} by qPCR assay. *P<0.05. n=3.

FIGS. 8A-8B (8A) Preferred internalization of EVs-VSMCs^{N1ICD} by HAoSMC at passage 2 compared with HAoSMC at passage 10 and human aortic endothelia cells (HAEC) in a co-culture system after 3 h incubation. EVs were labeled with PKH26 dye. WGA was used for cell boundary visualization. (8B) Quantitation of EVs internalization (PKH26 intensity normalized with cell numbers). ****P<0.0001, n=8. A.U.: arbitrary unit.

FIG. 9 In vivo uptake of EVs-VSMCs^{N1ICD} (50 μg) by aorta VSMCs from 22M old mice injected with EVs via tail vein. White arrows indicate uptaken EVs labeled with PKH26 dye in VMSCs (α-SMA positive cells). n=3. α-SMA: α smooth muscle actin.

FIGS. 10A-10E (10A) Representative fluorescence images of p16^{Ink4a} and γH2AX staining in female HAoSMC at passage 10 with different EVs (20 μg) treatment for 5 days. White arrows indicate positive stained cells. (10B) Quantitation of positive p16^{Inka} and γH2AX cells (n=4). * vs. PBS Ctrl, P<0.05; # vs. EVs-VSMCs^{TGFβ}, P<0.05. (10C) PKH-26 labeled EVs internalization by P10 female HAoSMC. (10D-10E) EVs from VSMCs treatment increased MIR503HG expression in female HAoSMC at P10 with higher copy number compared to PBS treated ones by RNAscope FISH assay. n=40-48 cells, P<0.001. Each dot represents one copy number. Ctrl: control; NC: negative control.

FIGS. 11A-11G show therapeutic effect of VSMCs^{N1ICD}-EVs on natural aging female mice. (11A) Schematic outline of in vivo EVs treatment. (11B) Representative images of aortic arch view with color doppler and aortic waveform. (11C) Aortic stiffness was evaluated by pulse wave velocity (PWV) in 22 months old female mice with either PBS or EVs from VSMCs^{N1ICD} treatments. EVs treatment significantly decreased thoracic PWV. n=6. (11D) Descending aortic peak velocity was decreased in mice with EVs treatment. n=6. (11E) Aortic calcification evaluated by Alizarin red in the aorta wall in 22M old female mice with PBS or EVs treatment. n=5. The black arrows indicate the positive staining. Immunofluorescent staining of γH2AX (11F) and p16^{Ink4a} (11G) in descending thoracic aortas from female old mice treated with PBS or EVs treatment and quantitative data. White arrows indicate positive VSMCs. n=5. Ctrl: control. A.U.: arbitrary unit.

FIG. 12 is an NMR spectra of synthesized DMPE-PEG-PD-L1-BP and PD-L1-BP.

FIG. 13A-13E are results showing a loss of MIR503HG in aged female VSMCs. (13A) a graph of MIR503HG relative expression at P2, P6, P8, and P10 in females and males. (13B) is a graph of Gm28730 relative expression in males and females 3M and 22M mice. (13C) is a graph of nuclear and cytosolic relative transcript abundance in NEAT1, 18S, and MIR503HG. (13D) are fluorescence images of MIR503HG visualization by RNA fluorescence in situ hybridization at female P2 and P10, and male P2 and P10. (13E) is a graph of MIR503HG copy for P2 and P10 in females and males.

FIG. 14A-14C are results showing MIR503HG deficiency induced cellular senescence in VSMCs. (14A) Representative fluorescence images of p16^{Ink4a} and γH2AX staining in growing and differentiated HAoSMC with MIR503HG KD treatment. (14B-14C) Quantitation of positive p16^{Ink4a} and γH2AX cells for Control versus MIR503HG KD in growing HAoSMC (14C) and differentiated HAoSMC (14C).

FIG. 15 are images showing human MIR503HG and mouse Gm28730 Gencode.

FIG. 16A-16C are graphs of MIR503HG relative expression for female (16A) and male (16B), and Gm28730 relative expression for male and female (16C).

FIG. 17A-17B are graphs of MIR503HG relative expression for female and male at P2, P6, P8, and P10 (17A). (17B) shows Gm28730 relative expression of male and female 3M and 22M mice.

Like reference symbols in the various drawings indicate like elements.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

### General Definitions

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. A range may be construed to include the start and the end of the range. For example, a range of 10% to 20% (i.e., range of 10%-20%) can includes 10% and also includes 20%, and includes percentages in between 10% and 20%, unless explicitly stated otherwise herein.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inactivate”, “inactivating” and “inactivation” means to decrease or eliminate an activity, response, condition, disease, or other biological parameter due to a chemical (covalent bond formation) between the ligand and a its biological target.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage. In particular, the term “treatment” includes the alleviation, in part or in whole, of the symptoms of coronavirus infection (e.g., sore throat, blocked and/or runny nose, cough and/or elevated temperature associated with a common cold). Such treatment may include eradication, or slowing of population growth, of a microbial agent associated with inflammation.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event. In particular embodiments, “prevention” includes reduction in risk of coronavirus infection in patients. However, it will be appreciated that such prevention may not be absolute, i.e., it may not prevent all such patients developing a coronavirus infection, or may only partially prevent an infection in a single individual. As such, the terms “prevention” and “prophylaxis” may be used interchangeably.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

### Biological Definitions

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers. In some embodiments, the polynucleotide is composed of nucleotide monomers of generally greater than 100 nucleotides in length and up to about 8,000 or more nucleotides in length.

Nucleic acids may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or chimeras or combinations thereof.

In some embodiments, polynucleotides of the present disclosure function as messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. The skilled artisan will appreciate that, except where otherwise noted, polynucleotide sequences set forth in the instant application will recite “T”s in a representative DNA sequence but where the sequence represents RNA (e.g., mRNA), the “T”s would be substituted for “U”s. Thus, any of the RNA polynucleotides encoded by a DNA identified by a particular sequence identification number may also comprise the corresponding RNA (e.g., mRNA) sequence encoded by the DNA, where each “T” of the DNA sequence is substituted with “U.”

The basic components of an mRNA molecule typically include at least one coding region, a 5′ untranslated region (UTR), a 3′ UTR, a 5′ cap and a poly-A tail. Polynucleotides of the present disclosure may function as mRNA but can be distinguished from wild-type mRNA in their functional and/or structural design features, which serve to overcome existing problems of effective polypeptide expression using nucleic-acid based therapeutics.

Polynucleotides of the present disclosure, in some embodiments, are codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art—non-limiting examples include services from GeneArt (Life Technologies), DNA2.0 (Menlo Park Calif.) and/or proprietary methods. In some embodiments, the open reading frame (ORF) sequence is optimized using optimization algorithms.

In some embodiments, a codon optimized sequence shares less than 95% sequence identity, less than 90% sequence identity, less than 85% sequence identity, less than 80% sequence identity, or less than 75% sequence identity to a naturally-occurring or wild-type sequence (e.g., a naturally-occurring or wild-type mRNA sequence encoding a polypeptide or protein of interest (e.g., an antigenic protein or antigenic polypeptide)).

In some embodiments, a codon-optimized sequence shares between 65% and 85% (e.g., between about 67% and about 85%, or between about 67% and about 80%) sequence identity to a naturally-occurring sequence or a wild-type sequence (e.g., a naturally-occurring or wild-type mRNA sequence encoding a polypeptide or protein of interest (e.g., an antigenic protein or polypeptide)). In some embodiments, a codon-optimized sequence shares between 65% and 75%, or about 80% sequence identity to a naturally-occurring sequence or wild-type sequence (e.g., a naturally-occurring or wild-type mRNA sequence encoding a polypeptide or protein of interest (e.g., an antigenic protein or polypeptide)).

In some embodiments a codon-optimized RNA (e.g., mRNA) may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

The term “polypeptide” refers to a compound made up of a single chain of D- or L-amino acids or a mixture of D- and L-amino acids joined by peptide bonds.

In some embodiments, a polypeptide is longer than 25 amino acids and shorter than 50 amino acids. The term “antigenic polypeptide” includes full length polypeptides/proteins as well as immunogenic fragments thereof (immunogenic fragments capable of inducing an immune response to an infection agent). Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer, or tetramer. Polypeptides may also comprise single chain or multichain polypeptides such as antibodies or insulin and may be associated or linked. Most commonly, disulfide linkages are found in multichain polypeptides. The term polypeptide may also apply to amino acid polymers in which at least one amino acid residue is an artificial chemical analogue of a corresponding naturally-occurring amino acid.

A “polypeptide variant” is a molecule that differs in its amino acid sequence relative to a native sequence or a reference sequence. Amino acid sequence variants may possess substitutions, deletions, insertions, or a combination of any two or three of the foregoing, at certain positions within the amino acid sequence, as compared to a native sequence or a reference sequence. Ordinarily, variants possess at least 50% identity to a native sequence or a reference sequence. In some embodiments, variants share at least 80% identity or at least 90% identity with a native sequence or a reference sequence.

In some embodiments “variant mimics” are provided. A “variant mimic” contains at least one amino acid that would mimic an activated sequence. For example, glutamate may serve as a mimic for phosphoro-threonine and/or phosphoro-serine. Alternatively, variant mimics may result in deactivation or in an inactivated product containing the mimic. For example, phenylalanine may act as an inactivating substitution for tyrosine, or alanine may act as an inactivating substitution for serine.

“Analogs” is meant to include polypeptide variants that differ by one or more amino acid alterations, for example, substitutions, additions or deletions of amino acid residues that still maintain one or more of the properties of the parent or starting polypeptide.

The present disclosure provides several types of compositions that are polynucleotide or polypeptide based, including variants and derivatives. These include, for example, substitutional, insertional, deletion and covalent variants and derivatives. The term “derivative” is synonymous with the term “variant” and generally refers to a molecule that has been modified and/or changed in any way relative to a reference molecule or a starting molecule.

As such, polynucleotides encoding peptides or polypeptides containing substitutions, insertions and/or additions, deletions and covalent modifications with respect to reference sequences, in particular the polypeptide sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal residues or N-terminal residues) alternatively may be deleted depending on the use of the sequence, as for example, expression of the sequence as part of a larger sequence that is soluble, or linked to a solid support.

“Substitutional variants” when referring to polypeptides are those that have at least one amino acid residue in a native or starting sequence removed and a different amino acid inserted in its place at the same position. Substitutions may be single, where only one amino acid in the molecule has been substituted, or they may be multiple, where two or more (e.g., 3, 4 or 5) amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Features” when referring to polypeptide or polynucleotide are defined as distinct amino acid sequence-based or nucleotide-based components of a molecule respectively. Features of the polypeptides encoded by the polynucleotides include surface manifestations, local conformational shape, folds, loops, half-loops, domains, half-domains, sites, termini and any combination(s) thereof.

As used herein when referring to polypeptides the term “domain” refers to a motif of a polypeptide having one or more identifiable structural or functional characteristics or properties (e.g., binding capacity, serving as a site for protein-protein interactions).

As used herein when referring to polypeptides the terms “site” as it pertains to amino acid based embodiments is used synonymously with “amino acid residue” and “amino acid side chain.” As used herein when referring to polynucleotides the terms “site” as it pertains to nucleotide based embodiments is used synonymously with “nucleotide.” A site represents a position within a peptide or polypeptide or polynucleotide that may be modified, manipulated, altered, derivatized or varied within the polypeptide-based or polynucleotide-based molecules.

As recognized by those skilled in the art, protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein having a length of 10, 20, 30, 40, 50, 60, 70, 80, 90, 100 or longer than 100 amino acids. In another example, any protein that includes a stretch of 20, 30, 40, 50, or 100 (contiguous) amino acids that are 40%, 50%, 60%, 70%, 80%, 90%, 95%, or 100% identical to any of the sequences described herein can be utilized in accordance with the disclosure. In some embodiments, a polypeptide includes 2, 3, 4, 5, 6, 7, 8, 9, 10, or more mutations as shown in any of the sequences provided herein or referenced herein. In another example, any protein that includes a stretch of 20, 30, 40, 50, or 100 amino acids that are greater than 80%, 90%, 95%, or 100% identical to any of the sequences described herein, wherein the protein has a stretch of 5, 10, 15, 20, 25, or 30 amino acids that are less than 80%, 75%, 70%, 65% to 60% identical to any of the sequences described herein can be utilized in accordance with the disclosure.

Polypeptide or polynucleotide molecules of the present disclosure may share a certain degree of sequence similarity or identity with the reference molecules (e.g., reference polypeptides or reference polynucleotides), for example, with art-described molecules (e.g., engineered or designed molecules or wild-type molecules). The term “identity,” as known in the art, refers to a relationship between the sequences of two or more polypeptides or polynucleotides, as determined by comparing the sequences. In the art, identity also means the degree of sequence relatedness between two sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. Identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al. (1997).” Gapped BLAST and PSI-BLAST: a new generation of protein database search programs,” Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently, a Fast Optimal Global Sequence Alignment Algorithm (FOGSAA) was developed that purportedly produces global alignment of nucleotide and protein sequences faster than other optimal global alignment methods, including the Needleman-Wunsch algorithm. Other tools are described herein, specifically in the definition of “identity” below.

As used herein, the term “homology” refers to the overall relatedness between polymeric molecules, e.g. between nucleic acid molecules (e.g. DNA molecules and/or RNA molecules) and/or between polypeptide molecules. Polymeric molecules (e.g. nucleic acid molecules (e.g. DNA molecules and/or RNA molecules) and/or polypeptide molecules) that share a threshold level of similarity or identity determined by alignment of matching residues are termed homologous. Homology is a qualitative term that describes a relationship between molecules and can be based upon the quantitative similarity or identity. Similarity or identity is a quantitative term that defines the degree of sequence match between two compared sequences. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). Two polynucleotide sequences are considered homologous if the polypeptides they encode are at least 50%, 60%, 70%, 80%, 90%, 95%, or even 99% for at least one stretch of at least 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered homologous if the proteins are at least 50%, 60%, 70%, 80%, or 90% identical for at least one stretch of at least 20 amino acids.

Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function in the course of evolution. “Paralogs” are genes (or proteins) related by duplication within a genome. Orthologs retain the same function in the course of evolution, whereas paralogs evolve new functions, even if these are related to the original one.

The term “identity” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12 (1), 387 (1984)), BLASTP, BLASTN, and FASTA Altschul, S. F. et al., J. Molec. Biol., 215, 403 (1990)).

The term “complementary” refers to the topological compatibility or matching together of interacting surfaces of a probe molecule and its target. Thus, the target and its probe can be described as complementary, and furthermore, the contact surface characteristics are complementary to each other.

The term “hybridization” refers to a process of establishing a non-covalent, sequence-specific interaction between two or more complementary strands of nucleic acids into a single hybrid, which in the case of two strands is referred to as a duplex.

The term “anneal” refers to the process by which a single-stranded nucleic acid sequence pairs by hydrogen bonds to a complementary sequence, forming a double-stranded nucleic acid sequence, including the reformation (renaturation) of complementary strands that were separated by heat (thermally denatured).

The term “melting” refers to the denaturation of a double-stranded nucleic acid sequence due to high temperatures, resulting in the separation of the double strand into two single strands by breaking the hydrogen bonds between the strands.

The term “target” refers to a molecule that has an affinity for a given probe. Targets may be naturally-occurring or man-made molecules. Also, they can be employed in their unaltered state or as aggregates with other species.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8 (1), p. 466-472, 1988); SV40 enhancer; and the intron sequence between exons 2 and 3 of rabbit β-globin (Proc. Natl. Acad. Sci. USA., Vol. 78 (3), p. 1527-31, 1981). It is appreciated by those skilled in the art that the design of the expression vector can depend on such factors as the choice of the host cell to be transformed, the level of expression desired, etc.

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

The term “expression cassette” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

A nucleic acid sequence is “heterologous” to a second nucleic acid sequence if it originates from a foreign species, or, if from the same species, is modified by human action from its original form. For example, a heterologous promoter (or heterologous 5′ untranslated region (5′UTR)) operably linked to a coding sequence refers to a coding sequence from a species different from that from which the promoter was derived, or, if from the same species, a coding sequence which is different from naturally occurring allelic variants (for example, the 5′UTR or 3′UTR from a different gene is operably linked to a nucleic acid encoding for a co-stimulatory molecule).

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or antigen binding fragment thereof” or “antibody or fragments thereof” encompasses chimeric antibodies and hybrid antibodies, with dual or multiple antigen or epitope specificities, and fragments, such as F(ab′)2, Fab′, Fab, Fv, sFv, scFv and the like, including hybrid fragments. Thus, fragments of the antibodies that retain the ability to bind their specific antigens are provided. For example, fragments of antibodies which maintain binding activity are included within the meaning of the term “antibody or antigen binding fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or antigen binding fragment thereof” are conjugates of antibody fragments and antigen binding proteins (single chain antibodies). Also included within the meaning of “antibody or antigen binding fragment thereof” are immunoglobulin single variable domains, such as for example a nanobody.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

As used herein, “immune effector cells” refers to cells capable of binding an antigen or a peptide and which mediate an immune response. These cells include, but are not limited to, T cells (include CD4+ and CD8+ T cells), B cells, monocytes, macrophages, NK cells and cytotoxic T lymphocytes (CTLs).

### Compositions

Described herein are extracellular vesicles including a targeting peptide and an active agent. In some embodiments, the targeting peptide targets senescent cells. In some embodiments, the targeting peptide can be a programmed death-ligand 1 binding peptide (PD-L1-BP) having at least 80% sequence identity to SEQ ID NO: 1. In some embodiments, the targeting peptide can be conjugated to a PEG-conjugated phospholipid derivative.

In some embodiments, the PEG-conjugated phospholipid derivative can include, but is not limited to cholesterol (CLS)-PEG-NH-maleimide,

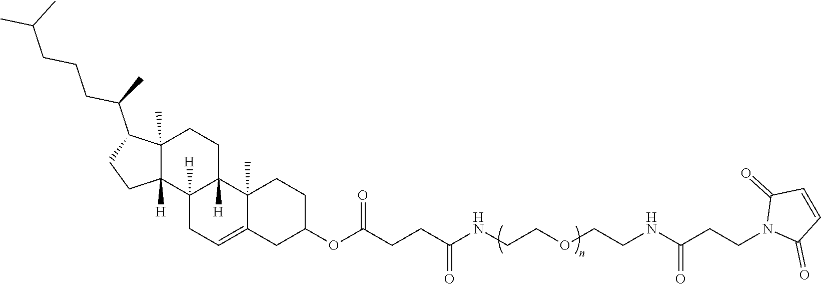

1,2-distearoyl-sn-glycero-3-phosphoethanolamine (DSPE)-PEG-maleimide,

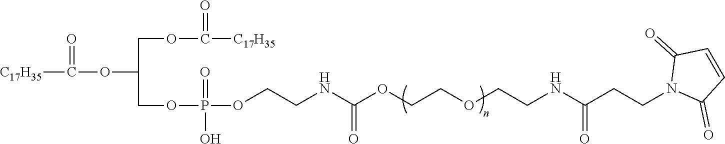

1,2-bis(diphenylphosphino) ethane (DPPE)-PEG-NH-maleimide,

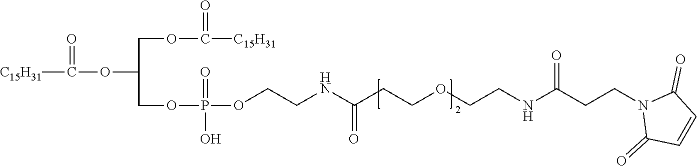

1,2-dilauroyl-sn-glycero-3-phosphoethanolamine (DLPE)-PEG-maleimide,

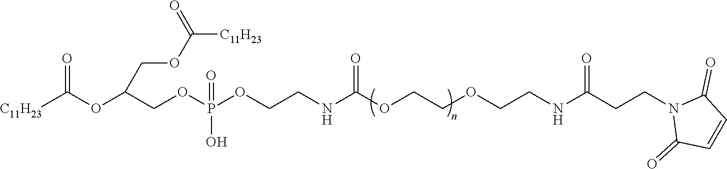

1,2-bis(dimethylphosphino) ethane (DMPE)-PEG-maleimide,

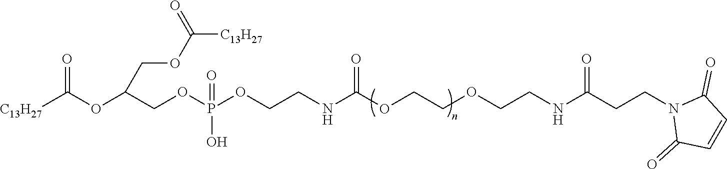

1,2-dioleoyl-sn-glycero-3-phosphoethanolamine (DOPE)-PEG-maleimide,

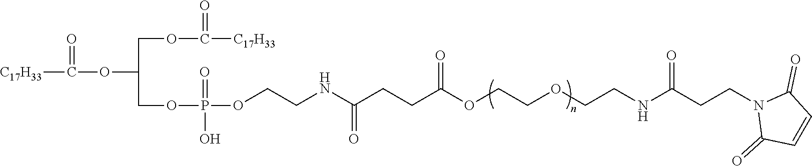

1,2-dipalmitoryl-sn-glycero-3-phosphoethanolamine (DPPE)-PEG-maleimide,

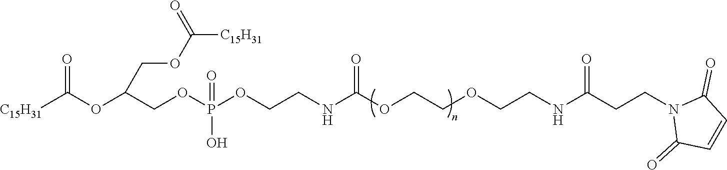

palmitic acid-PEG-maleimide,

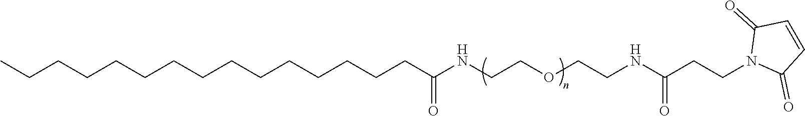

stearic acid (STA)-PEG-maleimide,

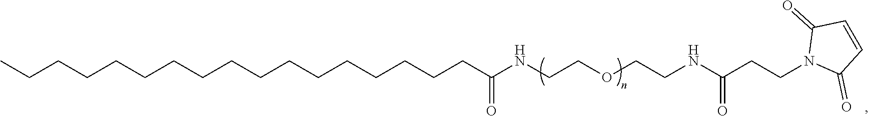

or any combination thereof.

In some embodiments, the PEG-conjugated phospholipid derivative can be DMPE-PEG-maleimide. In some embodiments, the PEG-conjugated phospholipid derivative can be DMPE-PEG5000-maleimide. In some embodiments, the targeting peptide conjugated to a PEG-conjugated phospholipid derivative can be DMPE-PEG-programmed death-ligand 1 binding peptide (PD-L1-BP). In some embodiments, the targeting peptide conjugated to a PEG-conjugated phospholipid derivative can be DMPE-PEG-PD-L1-BP, where the programmed death-ligand 1 binding peptide has at least 80% sequence identity to SEQ ID NO: 1.

In some embodiments, the extracellular vesicle is an extracellular vesicle derived from smooth muscle cells. In some embodiments, the smooth muscle cells can include, but are not limited to, vascular smooth muscle cells (VSMCs), bronchial/tracheal smooth muscle cells, lung smooth muscle cells, aorta smooth muscle cells, bladder smooth muscle cells, coronary artery smooth muscle cells, pulmonary artery smooth muscle cells, uterine smooth muscle cells, or any combination thereof. In some embodiments, the smooth muscle cells can be vascular smooth muscle cells (VSMCs). In some embodiments, the vascular smooth muscle cells (VSMCs) can be contractile vascular smooth muscle cells (e.g., mature vascular smooth muscle cells (VSMCs)).

In some embodiments, the extracellular vesicle can be an extracellular vesicle derived from vascular smooth muscle cells (VSMCs) from human induced pluripotent stem cells (hiPSC). In some embodiments, the human induced pluripotent stem cells (hiPSC) are induced by Notch1 intracellular domain (N1ICD) activation. In some embodiments, the human induced pluripotent stem cells (hiPSC) are induced by TGF-β activation.

In some embodiments, the active agent is a diagnostic, prophylactic, or therapeutic agent. In some embodiments, the active agent is a therapeutic agent. In some embodiments, the active agent can be a prophylactic agent. In some embodiments, the active agent can be a diagnostic agent. In some embodiments, the active agent can be anti-senescence active agent. In some embodiments, the anti-senescence active agent can be a nucleic acid, a protein/peptide, a small molecule, or any combination thereof. In some embodiments, the anti-senescence active agent can include, but are not limited to, metformin, resveratrol, rapalogs (agents related to rapamycin, dasatinib, quercetin, enzastaurin, fisetin, 17-DMAG, navitoclax, catechins, panobinostat, FOXO4-DRI. In some embodiments, the anti-senescence agent described herein can alter at least one signaling pathway of a src kinase, Akt kinase, ERK MAPK, p38 MAPK, histone deacetylase (HDAC), polar auxin transporter, monamine oxidase (MAO), protein kinase C-beta, calcineurin, and calmodulin.

In some embodiments, the anti-senescence active agent can be a nucleic acid. In some embodiments, the nucleic acid can include but is not limited to double stranded DNA, single-stranded DNA, complexed DNA, encapsulated DNA, naked RNA, encapsulated RNA, messenger RNA (mRNA), tRNA, short interfering RNA (siRNA), double stranded RNA (dsRNA), micro-RNA (miRNA), a long non-coding RNAs (lncRNAs), antisense RNA (asRNA), self-amplify mRNA (saRNA), guide RNA (gRNA), CRNA, or any combination thereof. In some embodiments, the nucleic acid can be a long non-coding RNAs. In some embodiments, the long non-coding RNAs (lncRNAs) is a MIR503HG the long non-coding RNAs (lncRNAs) having at least 80% sequence identity to SEQ ID NO: 2, MIR143HG the long non-coding RNAs (lncRNAs) having at least 80% sequence identity to SEQ ID NOs: 3 or 4, or any combination thereof. In some embodiments, the long non-coding RNAs (lncRNAs) is a MIR503HG the long non-coding RNAs (lncRNAs) having at least 80% sequence identity to SEQ ID NO: 2.

In some embodiments, the nucleic acid can be a micro-RNAs. In some embodiments, the micro-RNAs (miRNAs) can include miR-143, miR-145, miR-503, or any combination thereof.

In some embodiments, a senescent cell is selected from a senescent fibroblast, a senescent pre-adipocyte, a senescent epithelial cell, a senescent chondrocyte, a senescent neuron, a senescent smooth muscle cell, a senescent mesenchymal cell, a senescent macrophage, and a senescent endothelial cell. In certain specific embodiments, the senescent cell is a senescent pre-adipocyte.

### Pharmaceutical Compositions

Described herein are also pharmaceutical composition including a pharmaceutically acceptable carrier and an effective amount of the extracellular vesicle described herein.

The term “pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy (Polyethylene glycol)-1000], 1,2-Distearoyl-sn-glycero-3-Phosphoethanolamine-N-[Methoxy (Polyethylene glycol)-2000], and 1,2-Distearoyl-sn-glycero-3-Phosphoethanolamine-N-[Methoxy (Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly(meth)acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

In some embodiments, the pharmaceutical compositions can be administered locally. In some embodiments, the compounds are incorporated in a delivery system such as gels, nanoparticles, microparticles, or implants such as (e.g., rods, discs, wafers, orthopedic implants) for sustained release. In some embodiments, the compounds can be administered using a local delivery implantable system comprising the compounds incorporated within a gel, nanoparticles, microparticles, or an implant. In some embodiments, the pharmaceutical compositions comprise a delivery system such as gels, nanoparticles, microparticles, or implants such as (e.g., rods, discs, wafers, orthopedic implants) for sustained release of the active agent or a pharmaceutically acceptable salt or derivative thereof.

### Methods of Use

Described herein are methods for delivering an active agent into a senescent cell, including: introducing into the senescent cell an extracellular vesicle described herein or a composition described herein.

Described herein are also methods for treating or preventing conditions, diseases, or disorders related to, associated with, or caused by cellular senescence in a subject in need thereof, the method including administering to a subject in need thereof an extracellular vesicle described herein or a composition described herein.

In some embodiments, the conditions, diseases, or disorders related to, associated with, or caused by cellular senescence can include senescent cell associated diseases and disorders.

In certain embodiments of the methods described herein, the senescent cell associated disease or disorder is not cancer. Senescent cell associated diseases and disorders can include, for example, neurological diseases and disorders (e.g., Parkinson's disease, mild cognitive impairment (MCI), motor neuron dysfunction (MND), Huntington's disease, and diseases and disorders of the eyes, such as the neurodegenerative disease/disorder, macular degeneration, diseases of the eye that are associated with increasing age are glaucoma, vision loss, and cataracts, Alzheimer's disease and other dementias); cardiovascular disease (e.g., angina, arrhythmia, atherosclerosis, cardiomyopathy, peripheral vascular disease congestive heart failure, coronary artery disease (CAD), carotid artery disease, endocarditis, heart attack (coronary thrombosis, myocardial infarction [MI]), high blood pressure/hypertension, hypercholesterolemia/hyperlipidemia, mitral valve prolapse, peripheral artery disease (PAD), aortic aneurysm, brain aneurysm, cardiac fibrosis, cardiac diastolic dysfunction, cardiac stress resistance, and stroke); metabolic diseases and disorders (e.g., obesity, diabetes, metabolic syndrome, diabetic ulcers); inflammatory diseases and disorders (e.g., osteoporosis, psoriasis, oral mucositis, rheumatoid arthritis, inflammatory bowel disease, eczema, kyphosis, herniated intervertebral disc, and certain fibrosis or fibrotic conditions of organs such as renal fibrosis, liver fibrosis, pancreatic fibrosis, cardiac fibrosis, skin wound healing, and oral submucous fibrosis); pulmonary diseases and disorders (e.g., idiopathic pulmonary fibrosis (IPF), chronic obstructive pulmonary disease (COPD), asthma, cystic fibrosis, bronchiectasis, and emphysema); dermatological disease or disorder (e.g., psoriasis, eczema, rhytides, pruritis (linked to aging), dysesthesia, psoriasis and other papulosquamous disorders, reactive neutrophilic dermatosis, pemphigus, pemphigoid and other immunobullous dermatosis, cutaneous lymphomas, and cutaneous lupus); cerebrovascular disease; age-related disease or disorder that occurs as part of the natural aging process or that occurs when the subject is exposed to a senescence inducing agent or factor (e.g., irradiation, chemotherapy, smoking tobacco, high-fat/high sugar diet, other environmental factors) (renal dysfunction, glomerular disease, kyphosis, herniated intervertebral disc, frailty, hair loss, hearing loss, vision loss (blindness or impaired vision), muscle fatigue, skin conditions, skin nevi, diabetes, metabolic syndrome, sarcopenia, wrinkles, including superficial fine wrinkles, hyperpigmentation, scars, keloid, dermatitis, psoriasis, eczema (including seborrheic eczema), rosacca; vitiligo, ichthyosis vulgaris, dermatomyositis, and actinic keratosis), benign prostatic hypertrophy).

A prominent feature of aging is a gradual loss of function, or degeneration that occurs at the molecular, cellular, tissue, and organismal levels. Age-related degeneration gives rise to well-recognized pathologies, such as sarcopenia, atherosclerosis and heart failure, osteoporosis, pulmonary insufficiency, renal failure, neurodegeneration (including macular degeneration, Alzheimer's disease, and Parkinson's disease), and many others. Although different mammalian species vary in their susceptibilities to specific age-related pathologies, collectively, age-related pathologies generally rise with approximately exponential kinetics beginning at about the mid-point of the species-specific life span (e.g., 50-60 years of age for humans) (see, e.g., Campisi, Annu. Rev. Physiol. 75:685-705 (2013); Naylor et al., Clin. Pharmacol. Ther. 93:105-16 (2013)).

The effectiveness of an anti-senescence agent for treating or preventing conditions, diseases, or disorders related to, associated with, or caused by cellular senescence, including age-related diseases and disorders in a subject in need thereof can readily be determined by a person skilled in the medical and clinical arts. One or any combination of diagnostic methods, including physical examination, assessment and monitoring of clinical symptoms, and performance of analytical tests and methods described herein and practiced in the art, may be used for monitoring the health status of the subject. The effects of the treatment can be analyzed using techniques known in the art, such as comparing symptoms of patients suffering from or at risk of an age-related disease or disorder (e.g., cardiovascular disease; neurological diseases and disorders; metabolic diseases and disorders; inflammatory diseases and disorders; pulmonary diseases and disorders; dermatological disease or disorder; cerebrovascular disease, or any combination thereof) that have received the treatment with those of patients without such a treatment or with placebo treatment.

Described herein are methods of treating or preventing vascular diseases, the method including administering to a subject in need thereof an extracellular vesicle described herein or a composition described herein. In some embodiments, wherein the vascular disease is a cardiovascular disease (e.g., angina, arrhythmia, atherosclerosis, cardiomyopathy, peripheral vascular disease congestive heart failure, coronary artery disease (CAD), carotid artery disease, endocarditis, heart attack (coronary thrombosis, myocardial infarction [MI]), high blood pressure/hypertension, hypercholesterolemia/hyperlipidemia, mitral valve prolapse, peripheral artery disease (PAD), aortic aneurysm, brain aneurysm, cardiac fibrosis, cardiac diastolic dysfunction, cardiac stress resistance, and stroke).

Described herein are also methods for reducing cellular senescence in human aortic smooth muscle cells (HAoSMC) by decreasing the expression of senescence cell associated molecules, the method including administering to a subject in need thereof an extracellular vesicle described herein or a composition described herein.

Described herein are also methods for reducing cellular senescence by restoring expression levels of MIR503HG in senescent smooth muscle cells, the method including administering to a subject in need thereof an extracellular vesicle including a therapeutically effective amount of a MIR503HG long non-coding RNAs (lncRNAs) having at least 80% sequence identity to SEQ ID NO: 2, and a targeting peptide, wherein the targeting peptide targets senescent cells.

In some embodiments, the targeting peptide can be a programmed death-ligand 1 binding peptide (PD-L1-BP) having at least 80% sequence identity to SEQ ID NO: 1. In some embodiments, the targeting peptide can be conjugated to a PEG-conjugated phospholipid derivative.

In some embodiments, the PEG-conjugated phospholipid derivative is selected from cholesterol (CLS)-PEG-NH-maleimide, 1,2-distearoyl-sn-glycero-3-phosphoethanolamine (DSPE)-PEG-maleimide, 1,2-bis(diphenylphosphino) ethane (DPPE)-PEG-NH-maleimide, 1,2-dilauroyl-sn-glycero-3-phosphoethanolamine (DLPE)-PEG-maleimide, 1,2-bis(dimethylphosphino) ethane (DMPE)-PEG-maleimide (e.g., DMPE-PEG5000-maleimide), 1,2-dioleoyl-sn-glycero-3-phosphoethanolamine (DOPE)-PEG-maleimide, 1,2-dipalmitoryl-sn-glycero-3-phosphoethanolamine (DPPE)-PEG-maleimide, palmitic acid-PEG-maleimide, and stearic acid (STA)-PEG-maleimide, or any combination thereof.

In some embodiments, the PEG-conjugated phospholipid derivative can be DMPE-PEG-maleimide. In some embodiments, the PEG-conjugated phospholipid derivative can be DMPE-PEG5000-maleimide. In some embodiments, the targeting peptide conjugated to a PEG-conjugated phospholipid derivative can be DMPE-PEG-programmed death-ligand 1 binding peptide (PD-L1-BP). In some embodiments, the targeting peptide conjugated to a PEG-conjugated phospholipid derivative can be DMPE-PEG-PD-L1-BP, where the programmed death-ligand 1 binding peptide has at least 80% sequence identity to SEQ ID NO: 1.

In some embodiments, the extracellular vesicle is an extracellular vesicle derived from smooth muscle cells. In some embodiments, the smooth muscle cells can include, but are not limited to, vascular smooth muscle cells (VSMCs), bronchial/tracheal smooth muscle cells, lung smooth muscle cells, aorta smooth muscle cells, bladder smooth muscle cells, coronary artery smooth muscle cells, pulmonary artery smooth muscle cells, uterine smooth muscle cells, or any combination thereof. In some embodiments, the smooth muscle cells can be vascular smooth muscle cells (VSMCs). In some embodiments, the vascular smooth muscle cells (VSMCs) can be contractile vascular smooth muscle cells (e.g., mature vascular smooth muscle cells (VSMCs)).

In some embodiments, the extracellular vesicle can be an extracellular vesicle derived from vascular smooth muscle cells (VSMCs) from human induced pluripotent stem cells (hiPSC). In some embodiments, the human induced pluripotent stem cells (hiPSC) are induced by Notch1 intracellular domain (N1ICD) activation. In some embodiments, the human induced pluripotent stem cells (hiPSC) are induced by TGF-β activation.

In some embodiments, a senescent cell is selected from a senescent fibroblast, a senescent pre-adipocyte, a senescent epithelial cell, a senescent chondrocyte, a senescent neuron, a senescent smooth muscle cell, a senescent mesenchymal cell, a senescent macrophage, and a senescent endothelial cell. In certain specific embodiments, the senescent cell is a senescent pre-adipocyte.

A senescent cell may exhibit any one or more of the following characteristics. (1) Senescence growth arrest is essentially permanent and cannot be reversed by known physiological stimuli. (2) Senescent cells increase in size, sometimes enlarging more than twofold relative to the size of non-senescent counterparts. (3) Senescent cells express a senescence-associated β-galactosidase (SA-β-gal), which partly reflects the increase in lysosomal mass. (4) Most senescent cells express p16INK4a, which is not commonly expressed by quiescent or terminally differentiated cells. (5) Cells that senesce with persistent DDR signaling harbor persistent nuclear foci, termed DNA segments with chromatin alterations reinforcing senescence (DNA-SCARS). These foci contain activated DDR proteins and are distinguishable from transient damage foci. DNA-SCARS include dysfunctional telomeres or telomere dysfunction-induced foci (TIF). (6) Senescent cells express and may secrete molecules associated with senescence, which in certain instances may be observed in the presence of persistent DDR signaling, which in certain instances may be dependent on persistent DDR signaling for their expression. (7) The nuclei of senescent cells lose structural proteins such as Lamin B1 or chromatin-associated proteins such as histones and HMGB1. See, e.g., Freund et al., Mol. Biol. Cell 23:2066-75 (2012); Davalos ct al., J. Cell Biol. 201:613-29 (2013); Ivanov ct al., J. Cell Biol. DOI: 10.1083/jcb.201212110, page 1-15; published online Jul. 1, 2013; Funayama et al., J. Cell Biol. 175:869-80 (2006)).

Senescent cells and senescent cell associated molecules can be detected by techniques and procedures described in the art. For example, the presence of senescent cells in tissues can be analyzed by histochemistry or immunohistochemistry techniques that detect the senescence marker, SA-beta galactosidase (SA-β gal) (see, e.g., Dimri et al., Proc. Natl. Acad. Sci. USA 92:9363-9367 (1995)). The presence of the senescent cell-associated polypeptide p16 can be determined by any one of numerous immunochemistry methods practiced in the art, such as immunoblotting analysis. Expression of p16 mRNA in a cell can be measured by a variety of techniques practiced in the art including quantitative PCR. The presence and level of senescence cell associated polypeptides (e.g., polypeptides of the SASP) can be determined by using automated and high throughput assays, such as an automated Luminex array assay described in the art (see, e.g., Coppe et al., PLoS Biol 6:2853-68 (2008)).

The presence of senescent cells can also be determined by detection of senescent cell-associated molecules, which include growth factors, proteases, cytokines (e.g., inflammatory cytokines), chemokines, cell-related metabolites, reactive oxygen species (e.g., H202), and other molecules that stimulate inflammation and/or other biological effects or reactions that may promote or exacerbate the underlying disease of the subject. Senescent cell-associated molecules include those that are described in the art as comprising the senescence-associated secretory phenotype (SASP, i.e., which includes secreted factors which may make up the pro-inflammatory phenotype of a senescent cell), senescent-messaging secretome, and DNA damage secretory program (DDSP). These groupings of senescent cell associated molecules, as described in the art, contain molecules in common and are not intended to describe three separate distinct groupings of molecules. Senescent cell-associated molecules include certain expressed and secreted growth factors, proteases, cytokines, and other factors that may have potent autocrine and paracrine activities (see, e.g., Coppe et al., supra; Coppe et al. J. Biol. Chem. 281:29568-74 (2006); Coppe et al. PLoS One 5:39188 (2010); Krtolica et al. Proc. Natl. Acad. Sci. U.S.A. 98:12072-77 (2001); Parrinello et al., J. Cell Sci. 118:485-96 (2005). ECM associated factors include inflammatory proteins and mediators of ECM remodeling and which are strongly induced in senescent cells (see, e.g., Kuilman et al., Nature Reviews 9:81-94 (2009)). Other senescent cell-associated molecules include extracellular polypeptides (proteins) described collectively as the DNA damage secretory program (DDSP) (see, e.g., Sun et al., Nature Medicine published online 5 Aug. 2012; doi: 10.1038/nm.2890). Senescent cell-associated proteins also include cell surface proteins (or receptors) that are expressed on senescent cells, which include proteins that are present at a detectably lower amount or are not present on the cell surface of a non-senescent cell.

Senescence cell-associated molecules include secreted factors which may make up the pro-inflammatory phenotype of a senescent cell (e.g., SASP). These factors include, without limitation, GM-CSF, GROα, GROα,β,γ, IGFBP-7, IL-1α, IL-6, 1L-7, 1L-8, MCP-1, MCP-2, MIP-1α, MMP-1, MMP-10, MMP-3, Amphiregulin, ENA-78, Eotaxin-3, GCP-2, GITR, HGF, ICAM-1, IGFBP-2, IGFBP-4, IGFBP-5, IGFBP-6, IL-13, IL-1β, MCP-4, MIF, MIP-3α, MMP-12, MMP-13, MMP-14, NAP2, Oncostatin M, osteoprotegerin, PIGF, RANTES, sgp130, TIMP-2, TRAIL-R3, Acrp30, angiogenin, Axl, bFGF, BLC, BTC, CTACK, EGF-R, Fas, FGF-7, G-CSF, GDNF, HCC-4, 1-309, IFN-γ, IGFBP-1, IGFBP-3, IL-1R1, IL-11, IL-15, IL-2R-α, TL-6R, I-TAC, Leptin, LIF, MMP-2, MSP-a, PAI-1, PAI-2, PDGF-BB, SCF, SDF-1, sTNF RI, sTNF R11, Thrombopoietin, TIMP-1, tPA, uPA, uPAR, VEGF, MCP-3, 1GF-1, TGF-β3, MIP-1-delta, IL-4, FGF-7, PDGF-BB, IL-16, BMP-4, MDC, MCP-4, IL-10, TIMP-1, Fit-3 Ligand, ICAM-1, Axl, CNTF, INF-γ, EGF, BMP-6. Additional identified factors, which include those sometimes referred to in the art as senescence messaging secretome (SMS) factors, some of which are included in the listing of SASP polypeptides, include without limitation, IGF1, IGF2, and IGF2R, IGFBP3, IDFBP5, IGFBP7, PAl1, TGF-β, WNT2, IL-1a, IL-6, IL-8, and CXCR2-binding chemokines. Cell-associated molecules also include without limitation the factors described in Sun et al., Nature Medicine, supra, and include, including, for example, products of the genes, MMP1, WNT16B, SFRP2, WP12, SPINK1, MMP10, ENPP5, EREG, BMP6, ANGPTL4, CSGALNACT, CCL26, AREG, ANGPT1, CCK, THBD, CXCL14, NOV, GAL, NPPC, FAM150B, CST], GDNF, MUCL1, NPTX2, TMEM155, EDN1, PSG9, ADAMTS3, CD24, PPBP, CXCL3, MMP3, CST2, PSG8, PCOLCE2, PSG7, TNFSFJ5, C17,91167, CALCA, FGF18, IL8, BMP2, MATN3, TFP1, SERPINI 1, TNFRSF25, and IL23A. In some embodiments, the senescence cell-associated molecules can include p21, γH2Ax, p16^{Ink4a} and SASP. Senescent cell-associated proteins also include cell surface proteins (or receptors) that are expressed on senescent cells, which include proteins that are present at a detectably lower amount or are not present on the cell surface of a non-senescent cell.

### Methods of Administration

The compositions as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. The active agent may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc. Administration of the active components of their compositions can be a single administration, or at continuous and distinct intervals as can be readily determined by a person skilled in the art.

In certain embodiments, it may be desirable to provide continuous delivery of one or more compounds to a patient in need thereof. For intravenous or intraarterial routes, this can be accomplished using drip systems, such as by intravenous administration. For topical applications, repeated application can be done or a patch can be used to provide continuous administration of the compounds over an extended period of time.

The active agent may be administered in such amounts, time, and route deemed necessary in order to achieve the desired result. The exact amount of the active agent will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The exact amount of an active agent required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Reference will now be made in detail to specific aspects of the disclosed materials, compounds, compositions, articles, and methods, examples of which are illustrated in the accompanying Examples and Figures.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims.

By way of non-limiting illustration, examples of certain embodiments of the present disclosure are given below.

SEQUENCE LISTING

| SEQ ID NO: | Description | Sequence |
|---|---|---|
| 1 | PD-L1-BP | CVRARTR |
| 2 | Homo sapiens MIR503 | gaaggtagaaggtggggtctgccggacgcgtgttcctgccaccaggt |
| | host gene (MIR503HG) | gcccgctccccgcgaggccggctcaggagcagaaggaagcccggt |
| | long non-coding RNA | gccagccagccttcctgaaagaccaagcccgcgccatccggcttcctc |
| | (NCBI Reference | cagtggacgcctgcaggacccaggaatgtttttcttgaaggcatccagc |
| | Sequence: NR_024607.1) | atctccagttagcagtactgattttttttccccccaacaaaggaacactac |
| | | atcaacactgttggggggacctggacacagaagactcctgtttcaag |
| | | aaaatacaatcatctctcaaaggctgtaatttatatgcattttaaaactcta |
| | | ggcattgaaaaccacccaagtgtcccaaatagaagggtaatatataatc |
| | | aatcactcagtgtaatattatacatcctttaaaaatgttattgggaaatgtttt |
| | | atgatctgtaagtccaaggaatcctctcccaccatttctttccccccgctg |
| | | ttcccccatacccacacttctttgttccaattggcatgtaaatttggttttcc |
| | | cgccaaatgagtcagtcatgatgggaacctcaactgatttgaacagatg |
| | | tgtgtcaatgttacttggaaaactagatgtcaataaccagggtcacagaa |
| | | aaaggcagtggtcacaactctgtaaaaatgtatgcatgcacacagaca |
| | | agaactaaagtggaaccccacacaggaaaacagtgggctgtactcca |
| | | gtgctgggacattgaatgactgtatgctgcttttgatttccgtta |
| 3 | Homo sapiens cardiac | agctcatataaggtaaaaggcagagtggcctctgtggccagggagcc |
| | mesoderm enhancer- | gcaggcaagggactaaggggaggggggctcagtgccagctgcttaa |
| | associated non-coding | aaatgcccctgtggcagcgaggggcaccagaggctgggtctaattag |
| | RNA (MIR143HG), | ttgagaagcagtgacacccccaaccactccccaaacaggctggctcc |
| | transcript variant 2, long | cgtctccaggccccaaggagccacacctggaccagaccccaggaaa |
| | non-coding RNA (NCBI | ggcaagggcgctagctggtgggagccaccccgccatgctgatgtcag |
| | Reference Sequence: | agaagcaagaactctggagaagcagcctcctgggaccagaggaggg |
| | NR_105060.1) | ccagcagcaggcagcccggagacagaactctcacccacgcctctcca |
| | | agcctcccaacagaagacagaggtcccccacagccagagacatttcc |
| | | tgaagacatggggaacacagaggcagaaacagcccatccacccagg |
| | | agctgtcccccacactgccgggagccggcacccagagccgccaggt |
| | | aaaactgaggccacctggttcaacatcacctttcacagaaggggaagc |
| | | agccacagaaagaagggcctcgttaagaagtggaacctgggacccc |
| | | caagcggtgtctctcatcctgactggggatccagagtaggagggagc |
| | | ctttggtggggttggagtcccgccacaggccaccagagcggagcagc |
| | | gcagcgccctgtctcccagcctgaggtgcagtgctgcatctctggtca |
| | | gttgggagtctgagatgaagcactgtagctcaggaagagagaagttgt |
| | | tctgcagccatcagcctggaagtgcctggctggtgggccttcttgcagt |
| | | agcttcccctggagaagaggaaaagcaaaccttcattgagacccaag |
| | | cggtctctcctgtgctctgtgacaataataaagttccagcccttggcaaa |
| | | aaaaaaa |
| 4 | Homo sapiens cardiac | agctcatataaggtaaaaggcagagtggcctctgtggccagggagcc |
| | mesoderm enhancer- | gcaggcaagggactaaggggaggggggctcagtgccagctgcttaa |
| | associated non-coding | aaatgcccctgtggcagcgaggggcaccagaggctgggtctaattag |
| | RNA (MIR143HG), | ttgagaagcagtgacacccccaaccactccccaaacaggctggctcc |
| | transcript variant 1, long | cgtctccaggccccaaggagccacacctggaccagaccccaggaaa |
| | non-coding RNA (NCBI | gacatgtggtggaaaccctacggccctggagcccaaggagagcaac |
| | Reference Sequence: | ggctgtaacaagtgcacaggcaagggcgctagctggtgggagccac |
| | NR_105059.1) | cccgccatgctgatgtcagagaagcaagaactctggagaagcagcct |
| | | cctgggaccagaggagggccagcagcaggcagcccggagacaga |
| | | actctcacccacgcctctccaagcctcccaacagaagacagaggtccc |
| | | ccacagccagagacatttcctgaagacatggggaacacagaggcag |
| | | aaacagcccatccacccaggagctgtcccccacactgccgggagcc |
| | | ggcacccagagccgccaggtaaaactgaggccacctggttcaacatc |
| | | acctttcacagaaggggaagcagccacagaaagaagggcctcgttaa |
| | | gaagtggaacctgggacccccaagcggtgtctctcatcctgactgggg |
| | | atccagagtaggagggagcctttggtggggttggagtcccgccacag |
| | | gccaccagagcggagcagcgcagcgccctgtctcccagcctgaggt |
| | | gcagtgctgcatctctggtcagttgggagtctgagatgaagcactgtag |
| | | ctcaggaagagagaagttgttctgcagccatcagcctggaagtgcctg |
| | | gctggtgggccttcttgcagtagcttcccctggagaagaggaaaagca |
| | | aaccttcattgagacccaagcggtctctcctgtgctctgtgacaataata |
| | | aagttccagcccttggcaaaaaaaaaa |

### EXAMPLES

### Example 1

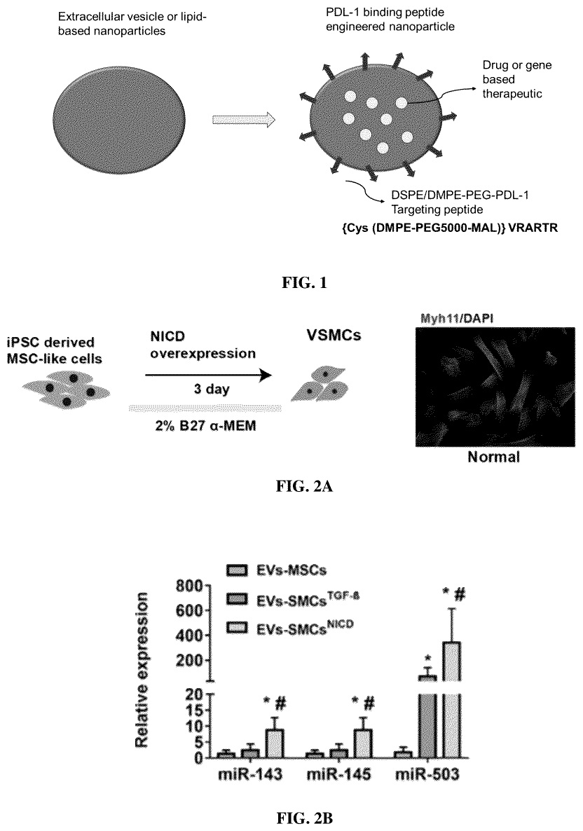

PD-L1 expression is directly related to increased expression in senescent cells both in vitro and in vivo (see Wang T W, et al., Nature. 2022; 611:358-364; Onorati A, et al., Mol Cell Biol. 2022; 42: e0017122). Senescent cells upregulate PD-L1 in p16 dependent manner (https://www.biorxiv.org/content/10.1101/2023.01.30.524522v1). Based on this, the extracellular vesicles or lipid-based nanoparticles with PDL-1 binding peptide were engineered for targeting delivery drug or gene-based therapeutics to the senescent cells in vivo. Overall, the goal is to develop drugs and gene-based targeting to overcome cellular senescence and offer protection. Successful targeting and delivery will provide benefits similar to anti-aging effects. The drug delivery and engineering-based details are shown in FIG. 1

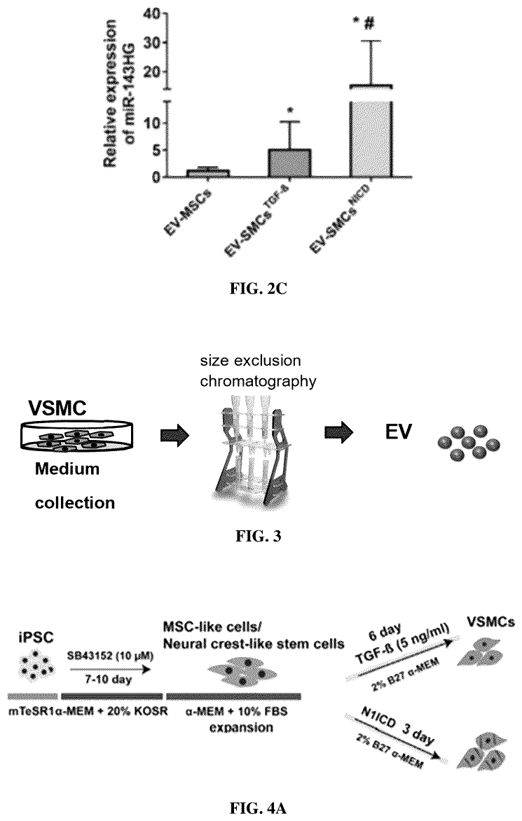

Specifically, it is of interest to engineer human contractile vascular smooth cells derived extracellular vesicles via the approach shown in FIG. 1 to target the senescent vascular smooth cells in vivo either during aging or vascular diseases. The procedures for generating PSC derived contractile vascular smooth muscle cells (VSMCs) and extracellular vehicles (EVs) are shown in FIGS. 2A-2B and FIG. 3.

### Targeting the Senescent Cells by PD-L1 Binding Peptide Engineered Nanoparticle

The goal is to develop drugs and gene-based targeting to overcome cellular senescence and offer protection. Successful targeting and delivery will provide benefits similar to anti-aging effects. Based on this, the extracellular vesicles (EVs) or lipid-based nanoparticles with programmed death-ligand 1 (PD-L1) binding peptide were engineered for targeting delivery drug or gene-based therapeutics to the senescent cells in vivo.

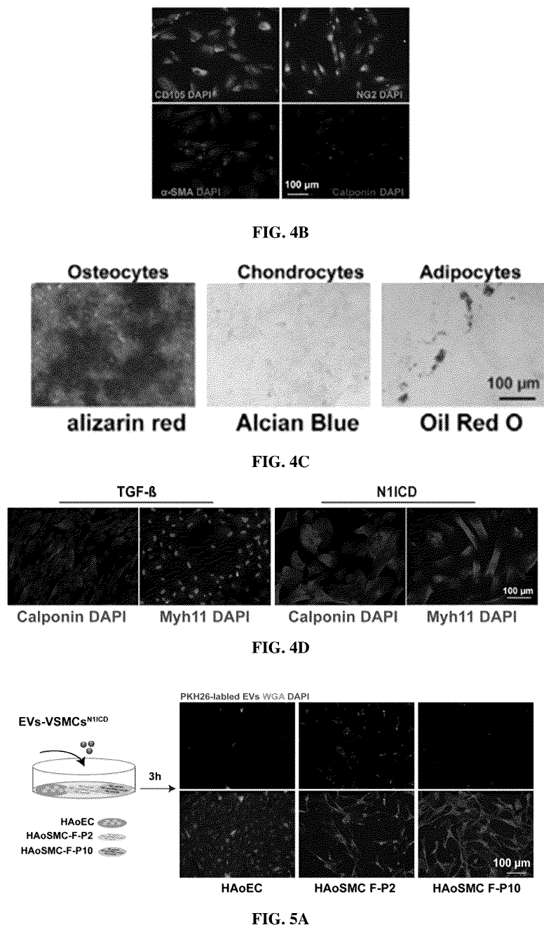

It is of interest to engineer human contractile vascular smooth cells derived extrasellar vesicles via the approach shown in FIG. 4A-4D to target the senescent vascular smooth cells in vivo either during aging or vascular diseases. The procedures for generating PSC derived contractile vascular smooth muscle cells (VSMCs) and EVs are shown in FIG. 4A-4D.

VSMCs generation: human induced pluripotent stem cells (hiPSC) were differentiated into mesenchymal stem-like cells (MSLCs)/neural crest like stem cells (NCLCs) using small molecule SB43152 (FIG. 4A). These cells expressed MSLC markers such as CD105 and NG2 (FIG. 4B) and could differentiate into osteocytes, chondrocytes, and adipocytes (FIG. 4C), then further differentiated them into VSMCs by using TGFβ or transiently activating Notch1 with the overexpression of Notch1 intracellular domain (N1ICD) (FIG. 4A). Transient activation of Notch1 successfully generated mature VSMCs with higher expression of contractile makers (FIG. 4D) for EVs production.

EVs generation: EVs isolation: EVs are isolated as previously described using size exclusion column (SEC) method (see Xuan W. et al., Stem Cell Res Ther. 2021; 12:131).

### PD-L1 Binding Peptide (PD-L1-BP) Engineering EVs:

PD-L1-BP [Cys-Val-Arg-Ala-Arg-Thr-Arg (CVRARTR) (SEQ ID NO: 1)] (see Moon Y. et al., Theranostics. 2022:12:1999-2014) (FIG. 12, synthesized by GenScript is covalently bound to DMPE-PEG5000-maleimide to synthesize DMPE-PEG-PD-L1-BP, which could be inserted into the membrane of EVs. EVs are incubated with 5-10 μM DMPE-PEG-PD-L1-BP at room temperature for 30 min to fabricate EVs-PD-L1-BP.

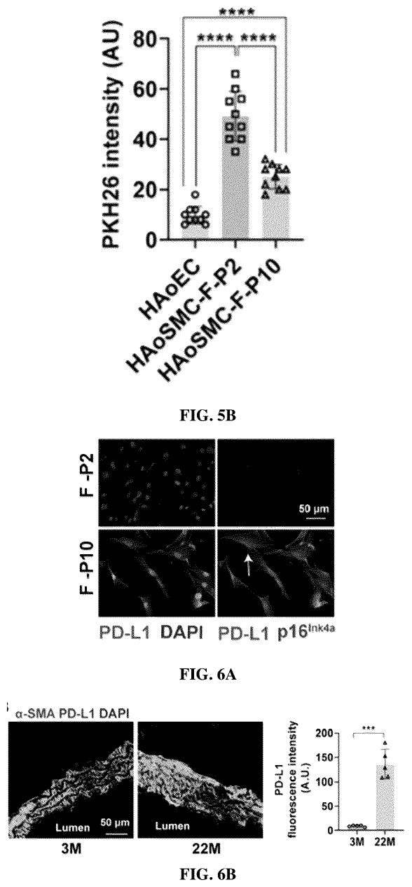

The surface components of EVs are partly responsible for their circulation kinetics and biodistribution (Murphy D E. et al., Exp Mol Med. 2019:51:1-12 (“Murphy, 2019”)). A previous study reported that intravenously administered EVs could be rapidly cleared from circulation in a partly macrophage-dependent manner (see Murphy, 2019; de Jong B. et al., Pharmaceutics. 2020:12). EVs from VSMCs^{N1ICD} showed preferential binding to VSMCs in vitro (FIGS. 5A-5B). However, it was noticed that the internalization of EVs was impaired in senescent VSMCs compared to non-senescent VSMCs (FIG. 5A-5B). Therefore, it is necessary to explore approaches to increase the circulation time of EVs and improve their delivery to the senescent VSMCs.

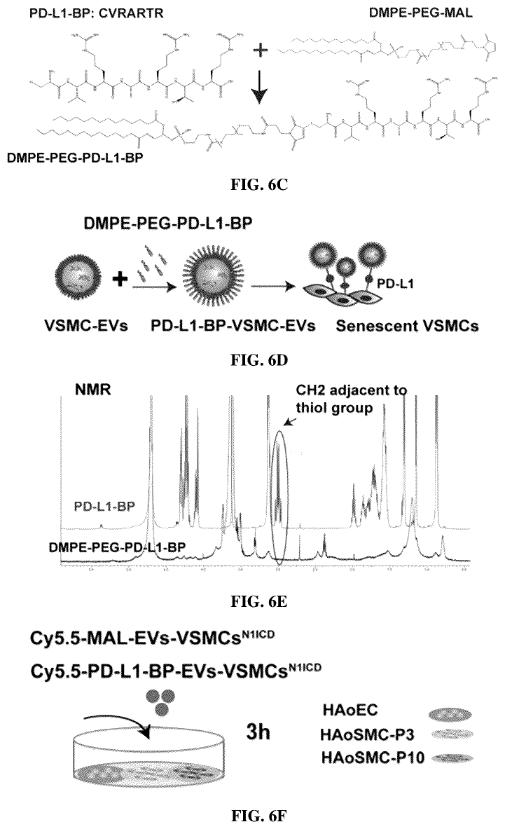

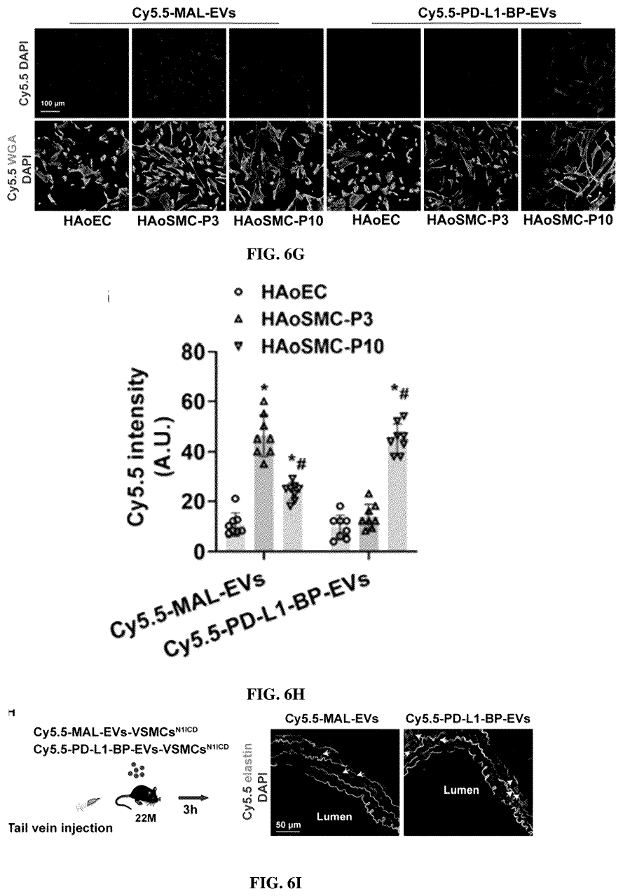

VSMCs to enhance therapeutic effects. EVs can be modified with targeting peptide to enhance their targeting ability and therapeutic effects. Polyethylene glycol (PEG), a hydrophilic polymer, is known to increase the circulation time of nanoparticles (see Suk J S. et al., Adv Drug Deliv Rev. 2016:99:28-51; Shi L. et al., Nanoscale. 2021:13:10748-10764). PEGylation increased the circulation time of EVs and reduced nonspecific interactions with cells (see Murphy D E. et al., Exp Mol Med. 2019:51:1-12). PD-L1, the immune checkpoint protein, is expressed by a broader range of cells (see Grabic N, et al., Circulation. 2007:116:2062-2071). PD-L1 expression was induced in VSMCs by inflammatory stimuli (see Koga N, et al., Arterioscler Thromb Vasc Biol. 2004:24:2057-2062). Recent studies reported that PD-L1 expression was upregulated in senescent cells (see Onorati A, et al., Mol Cell Biol. 2022; 42: e0017122; Wang T W, et al., Nature. 2022:611:358-364). The enhanced PD-L1 expression in senescent cells was in a p16^{Ink4a} dependent manner in aging and chronic inflammation (see Julia Majewska A A, et al., bioRxiv. 2023.01.30.524522). However, it remains unknow the expression pattern of PD-L1 in VSMCs during aging. PD-L1 expression was significantly upregulated both in replicative senescent HAoSMC (FIG. 6A) and in the aorta from aged mice (FIG. 6B). Therefore, modifying the surface of EVs to target PD-L1 could possibly enhance the delivery efficacy to senescent VSMCs. To further explore this possibility, the surface of VSMCs^{N1ICD} derived EVs was modified with PD-L1 binding peptide (PD-L1-BP, CVRARTR (SEQ ID NO: 1) by conjugating PD-L1-BP with a PEG-conjugated phospholipid derivative (DMPE-PEG) (FIG. 6C) to fabricate PD-L1-BP engineered EVs (PD-L1-BP-EVs) with PD-L1 targeting ability (FIG. 6D). Nuclear magnetic resonance (NMR) results confirmed the successful conjugation of PD-L1-BP and DMPE-PEG (FIG. 6E). cyanine 5.5 (Cy5.5) dye was also conjugated on PDL-BP conjugates for visualization. EVs modified with Cy5.5-PD-L1-BP were referred to as Cy5.5-PD-L1-BP-EVs. The conjugate of Cy5.5-DMPE-PEG (Cy5.5-PEG) was used as a control. EVs modified with Cy5.5-PEG were referred to as Cy5.5-PEG-EVs. Using a co-culture system with early and late passages of HAoSMC and HAoEC (FIGS. 6F-6G), PD-L1-BP-engineered EVs had better internalization with higher Cy5.5 intensity in replicative senescent HAoSMC compared with Cy5.5-PEG-EVs (FIG. 6H). Furthermore, PD-L1-BP-engineered EVs also exhibited higher uptake (higher Cy5.5 intensity) in the aorta from old mice 3 hours after systemic delivery (FIGS. 6I-6J). These preliminary results indicate that engineering EVs with PD-L1-BP could improve their targeting efficiency to senescent VSMCs.

Modifications to lipid-based nanoparticles can be employed in a similar manner to target senescent cells.

### Example 2

Vascular aging is closely associated with cardiovascular morbidity and mortality (Qiu Y, et al., J Transl Int Med. 2021:9:17-23). Evidence supports that the progression of aging related arterial stiffness and the associated risk of cardiovascular disease (CVD) differ between sexes (DuPont J J, et al., Br J Pharmacol. 2019:176:4208-4225). The onset of menopause coincides with accelerated vascular aging, suggesting that sex hormones influence the vascular aging (Armeni E, et al., Ther Adv Endocrinol Metab. 2022:13:20420188221129946). The increased risk of CVD in aged women may be linked to the deprivation of estrogens, leading to a loss of estrogen-mediated vascular protection (Xing D, et al., Arterioscler Thromb Vasc Biol. 2009; 29:289-295). However, chronic administration of estrogen has been associated with increased risks of cancer and cardiovascular diseases (Rossouw J E, et al., JAMA. 2002; 288:321-333; and D'Alonzo M, et al., Medicina (Kaunas). 2019; 55). Consequently, it is crucial to understand how vascular aging affects changes in estrogen mediated vascular protection and to identify novel therapeutic targets to preserve cardiovascular health in postmenopausal women.

With aging, vascular smooth muscle cells (VSMCs) undergo senescence, lose their contractile phenotype, and develop proinflammatory state (Gardner S E. et al., Arterioscler Thromb Vasc Biol. 2015:35:1963-1974). The accumulation of senescent VSMCs leads to inflammation, impairs arterial function, and promotes the onset of age-related diseases (Chi C. Li D J, et al., Biochim Biophys Acta Mol Basis Dis. 2019:1865:1810-1821). The long non-coding RNAs (lncRNAs) are crucial regulators of VSMCs phenotype (Zhong J Y, et al., Ann N Y Acad Sci. 2020; 1474:61-72; Das S, Zhang E, et al., Circ Res. 2018; 123:1298-1312; Leeper N J, et al., Cardiovasc Res. 2018; 114:611-621; and Cui X Y, et al., Ageing Res Rev. 2021; 72:101480). The lncRNA, MIR503HG is involved in cell differentiation, proliferation, and plasticity (Han X. et al., Biomed Pharmacother. 2023:160:114314). A reduction in MIR503HG expression was observed in the aortic tissue of patients with aneurysms, suggesting a potential role for MIR503HG in vascular biology (Tian Y. et al., Mediators Inflamm. 2023:2023:4003618). Interestingly a reduction in MIR503HG expression was observed in both ovarian cancer (commonly seen in postmenopausal women) and estrogen receptor-negative breast cancer cells and tissues, suggesting a potential association between MIR503HG and female hormones (Tian J, et al., Aging (Albany NY). 2022; 14:5390-5405; Doubeni C A, et al., Am Fam Physician. 2016; 93:937-944; and Fu J, et al., J Cell Mol Med. 2019; 23:4738-4745). Importantly, using in vitro replicative senescence model and biological aging mouse model, first showed a sex dependent expression pattern of MIR503HG in VSMCs with aging. The study suggested that MIR503HG is responsive to estrogen. 17-beta-estradiol (E2) triggered an increase in MIR503HG expression in early passage cultured VSMCs from both sexes. However, this effect was abolished by estrogen receptor α (ERα) antagonist. Interestingly, the E2-induced increase in MIR503HG expression was less evident in senescent female VSMCs, but not in their male counterparts. Furthermore, a decrease in transcriptional activation with E2 treatment in senescent female VSMCs using a luciferase reporter assay for estrogen response elements (EREs) was shown, which correlated with the expression pattern of MIR503HG. Importantly, the knockdown of MIR503HG led to a senescence-like phenotype in VSMCs from both sexes. In a preliminary work, extracellular vesicles (EVs) from bioengineered VSMCs were highly enriched with MIR503HG which effectively mitigated the senescent phenotype of aged female VSMCs. Hence, age-related decline of estrogens/ERα agonism in female was hypothesize, especially during the menopausal transition, leads to a dramatic decrease of MIR503HG expression in VSMCs which accelerates vascular aging in postmenopausal women. Restoration of MIR503HG in senescent VSMCs using EVs modified with targeting moieties will potentially improve vascular function of postmenopausal women. Therefore, the proposed hypotheses can be tested as follows.

(I) Test that disruption of estrogen/ERα-MIR503HG signaling leads to vascular senescence. replicative senescent VSMCs, biological aging and ovariectomized mice can be used to determine the role of estrogen/ERα-MIR503HG signaling pathway in the onset of vascular senescence and to elucidate the molecular mechanisms by which MIR503HG regulates VSMCs homeostasis.

RNA-binding proteins will be analyzed using Western Blot with the anti-HuR antibody. For competitive RNA pulldown assays, the HAoSMC cell lysates will be incubated with the same amount of biotinylated MIR503HG transcript along with variable amounts of nonbiotinylated oligonucleotides (0-1 μM) containing AREs sequence, and the expression of HuR in the RNA binding proteins will be analyzed.

RNA-Binding Protein Immunoprecipitation (RIP): RIP experiment will be conducted using Magna RIP™ RNA-Binding Protein Immunoprecipitation (RIP) Kit (Millipore Sigma) as described 52. Primary AoSMC at passage 2 (P2) will be used for this assay. Enrichment of MIR503HG will be analyzed by RT-PCR assay.

Ovariectomy in mice and E2 treatment: Ovariectomy will be performed in 3 months old young female mice as previously described 53. For E2 treatment, OVX mice or aged female mice will receive either 21-day time-release pellets containing 0.1 mg of E2 (Innovative Research of America), or placebo pellets that do not contain E2. These pellets will be implanted subcutaneously through an approximately 3-mm incision made on the dorsal aspect of the neck.

Expected results: enhanced expression of MIR503HG in non-senescent HAoSMC and mAoSMC from both sexes with estrogen exposure is anticipated, and ERα antagonist will be able to block this effect. Based on the initial findings, a female sex-biased reduction of the estrogenic effects and loss of MIR503HG expression in senescent/aged VSMCs is predicted. Knockdown of ERα in early passage HAoSMC will decrease the MIR503HG expression. According to a previous study⁴², ERα expression may decrease in female HAoSMC during replicative senescence compared to their male counterparts. It is possible that there are no significant changes in ERα expression. However, the reduced estrogenic response in senescent female VSMCs could potentially influence the expression of MIR503HG. For in vivo study, OVX is expected to decrease the MIR503HG expression in young mouse aortas, and E2 supplementation should be able to restore its expression level. However, supplementation with E2 might partially restore aortic MIR503HG expression or it does not have effects on MIR503HG expression in aged female VSMCs due to the loss of estrogenic response. Silencing MIR503HG in HAoSMC and mHAoSMC from both sexes will induce cellular senescence which will be confirmed by an increased expression of p16^{Ink4a} and γH2AX, and extensive release of SASP factors is also expected. According to preliminary data, MIR503HG is lost in replicative senescent female HAoSMC, and therefore it is predicted that restoring the expression level of MIR503HG in senescent female VSMCs could mitigate cellular senescence. On the contrary, the study showed no significant decline in MIR503HG expression in male senescent HAoSMC. Therefore, it is anticipated that MIR503HG overexpression may not have significant effects on cellular senescence in these cells. It is also anticipate that MIR503HG physically binds to HuR as will be confirmed by RNA pull-down and RIP assays. In addition, the experiments with competitive RNA pulldown assay will show a dose-dependent inhibition of HuR binding to biotinylated MIR503HG by increasing concentrations of unbiotinylated AREs RNA oligonucleotides. Knockdown of MIR503HG will promote the nuclear-cytoplasmic shuttling of HuR leading to excessive accumulation of HuR in the cytoplasm, while the depletion of HuR will counteract the cellular senescence induced by MIR503HG silencing. These results will support the hypothesis that the functional role of MIR503HG in VSMCs hemostasis is mediated by interaction with HuR. Other predicted binding proteins (Table 1) or perform RNA pulldown assays and mass spectrometry (MS) to identify MIR503HG interacting proteins to test the hypothesis can also be tested.

TABLE 1
Predicted binding proteins with MIR503HG

| Proteins | RF Classifier |
|---|---|
| HuR | 0.85 |
| hnRNPA1 | 0.85 |
| HnRNPA2B1 | 0.7 |
| MYOCD | 0.65 |

(II) Test that bioengineered extracellular vesicles enriched with MIR503HG alleviate vascular stiffness and senescence in aged females.

(IIA) Test the therapeutic effects of extracellular vesicles derived from mature VSMCs on vascular senescence in aged females.

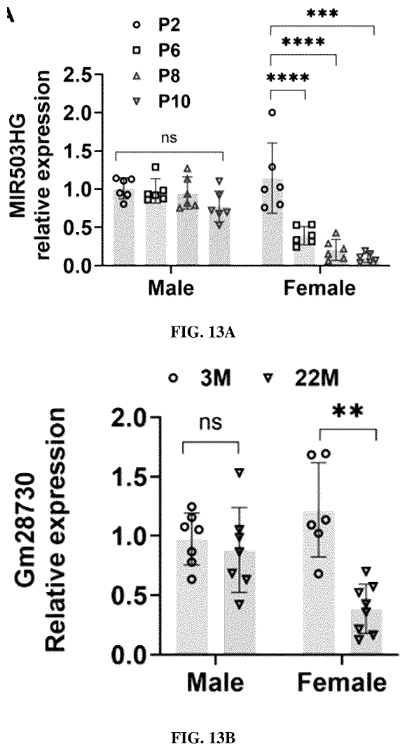

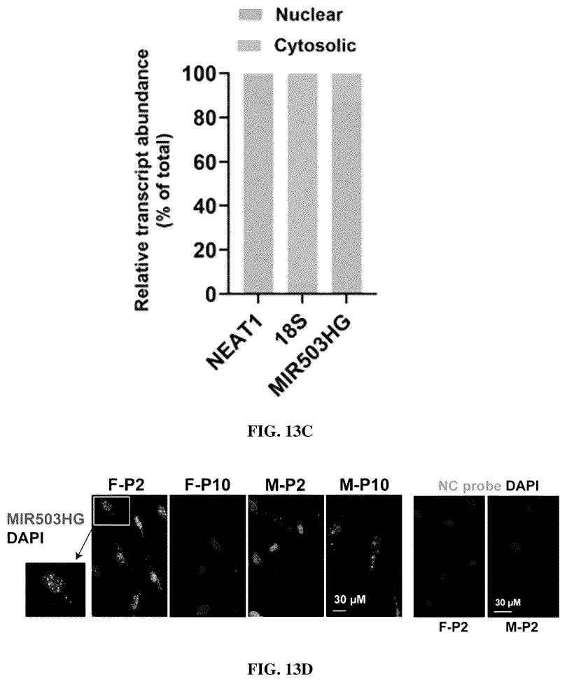

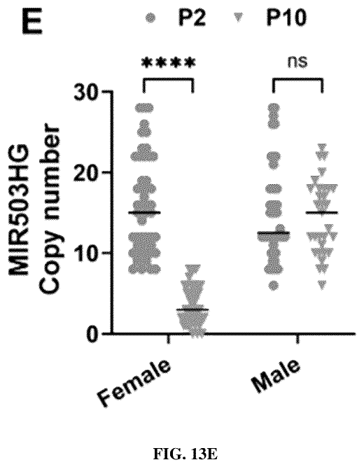

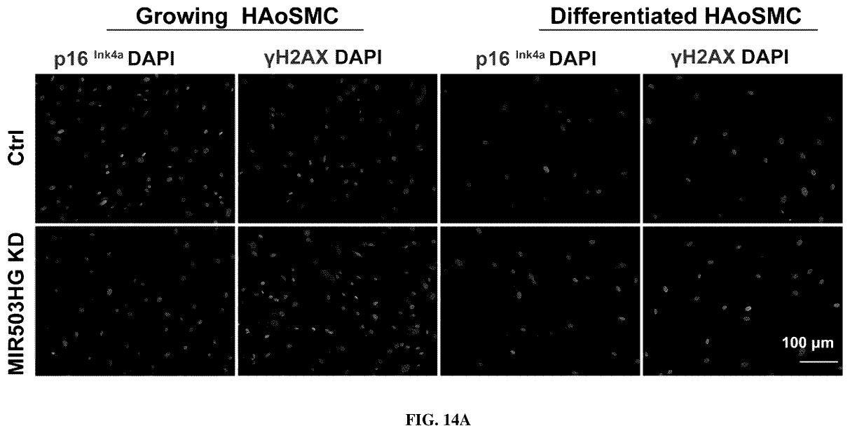

Preliminary data: the pilot study reported a loss of MIR503HG in aged female VSMCs (FIGS. 13A-13E).

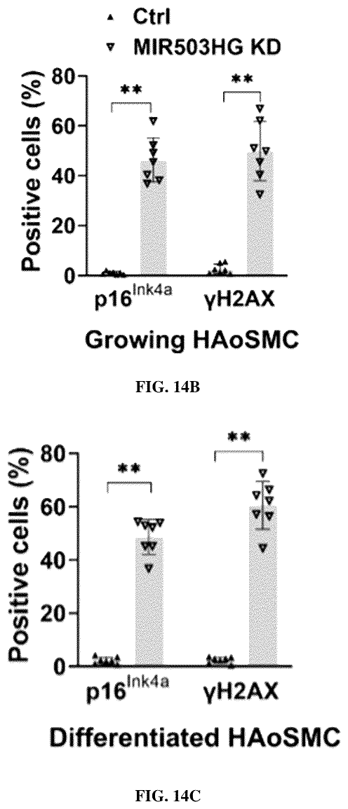

MIR503HG deficiency induced cellular senescence in VSMCs (FIG. 14A-14C).

Therefore, by restoring the MIR503HG level in the VSMCs of aged females could potentially alleviate their cellular senescence. However, overexpressing MIR503HG in VSMCs in vivo may present challenges. While viral vectors could be considered, safety concerns and issues with targeting efficiency make them less than ideal. Therefore, an alternative approach that offers high efficiency, low immune reaction and precise cell targeting is highly desirable.

Extracellular vesicles (EVs) are nanovesicles that are produced and released by cells and contain various components such as cytosolic proteins, lipids, and genetic factors including lncRNAs (Abels E R, et al., Cell Mol Neurobiol. 2016; 36:301-312). The intercellular transfer of lncRNA naturally occurs in EVs. In addition, EVs present significant advantages such as minimal immunogenicity, a reduced rate of degradation, and low cytotoxicity (Garbo S, et al., Int J Mol Sci. 2022; 23, doi: 10.3390/ijms23137471). Therefore, EVs could serve as a promising delivery vehicle for lncRNA-based therapeutics (Cecchin R, et al., Mol Ther. 2023; 31:1225-1230). Furthermore, EVs possess valuable innate properties from parent cells, they can cause phenotypic alterations in the recipient cells (Thakur A, et al., Protein Cell. 2022; 13:631-654). Studies have shown that EVs are preferentially taken up by cells of their own origin (Jurgielewicz B J, et al., Nanoscale Res Lett. 2020; 15:170; and Luengo-Mateos M, et al., STAR Protoc. 2024:5:102910). Thus, for therapeutic EVs production the parent cells need to be selected on the basis of their activity and targeted tissue/cell homing properties. Mature VSMCs, which exhibit a contractile phenotype, are the predominant cells in blood vessels and play a crucial role in maintaining their normal physiological function (Tang H Y, et al., Cells. 2022; 11, doi: 10.3390/cells11244060). EVs from mature VSMCs may inherently possess properties for maintaining contractile phenotype and homeostasis, similar to their parent cells. Importantly, higher MIR503HG expression level is found in differentiated HAoSMC than in proliferating ones (FIG. 5A). However, the limited availability of primary mature VSMCs for EVs production poses a significant challenge for translational applications.

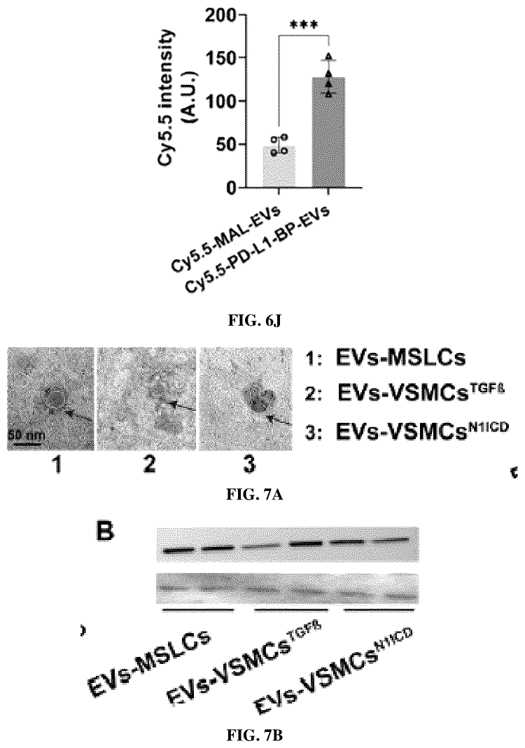

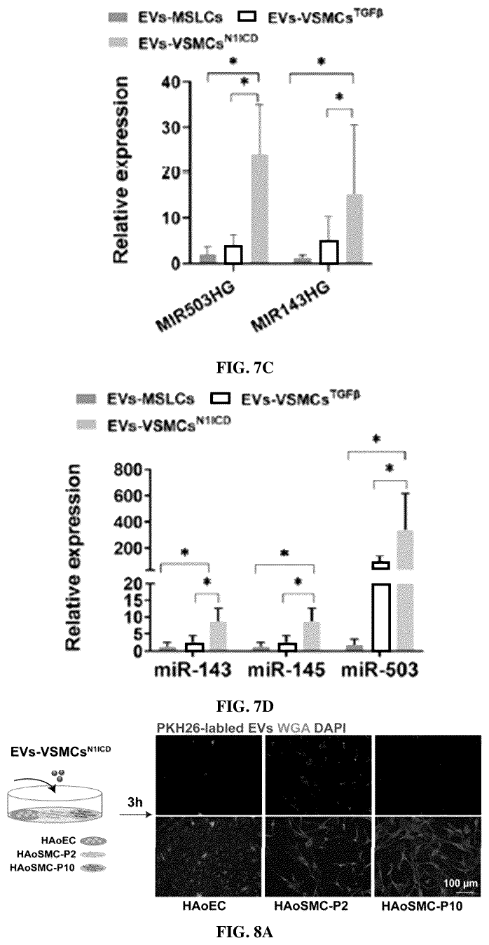

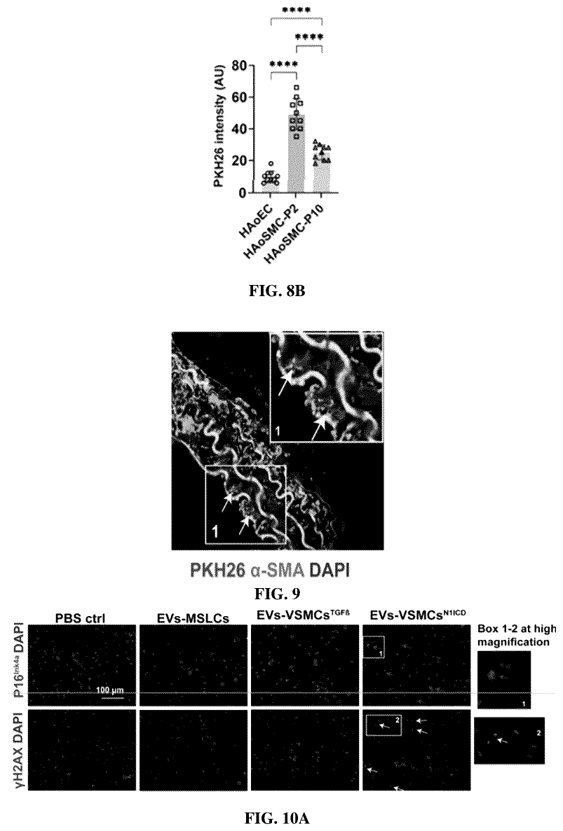

Human induced pluripotent stem cells (hiPSCs) possess multipotent differentiation potential, providing an unlimited source of specific parent cells for the production of EVs (Di Baldassarre A, et al., Cells. 2018; 7, doi: 10.3390/cells7060048). In the study, differentiated hiPSCs into VSMCs using a two-step approach was successfully. Initially, hiPSCs were induced into mesenchymal stem-like cells (MSLCs) using a small molecule SB43152 (FIG. 4A). These cells expressed MSLC markers such as CD105 and NG2 (FIG. 4B) and demonstrated the ability to differentiate into osteocytes, chondrocytes, and adipocytes (FIG. 4C). Subsequently, these cells were differentiated into VSMCs using TGFβ or through transient activation of Notch1 by overexpressing Notch1 intracellular domain (N1ICD) (FIG. 4A). These iPSC derived VSMCs are referred to as VSMCs^{TGFβ} and VSMCs^{N1ICD}, respectively. Compared with the traditional TGFβ induction method, the transient activation of Notch1 resulted in the generation of mature VSMCs with higher expression of contractile markers (FIG. 4D). EVs from MSLCs, VSMCs differentiated by either TGFβ or Notch1 activation showed similar morphology by transmission electron microscopy (TEM) (FIG. 7A) and expressed EV markers (CD9 and CD63) (FIG. 7B). However, EVs derived from mature VSMCs, induced by Notch1 activation, were significantly enriched with lncRNAs (MIR143HG and MIR503HG) (FIG. 7C) and miRNAs (miR-143, miR-145, miR-503) (FIG. 7D). These lncRNAs and miRNAs are known to regulate contractility and hemostasis in VSMCs (Dong K, et al., Circulation. 2021; 144:1856-1875). To further support the rationale, a co-culture system of primary HAoSMC at P2 and P10, along with human aortic endothelial cells (HAEC) was utilized to investigate the preferential uptake of EVs from mature VSMCs. After incubating with 10 μg of EVs-VSMCs^{N1ICD} for 3 hours, the majority of the EVs were internalized by the HAoSMC, not by the HAEC (FIG. 8A), suggesting a preferential uptake of EVs by VSMCs. Furthermore, VSMCs at early passage demonstrated increased uptake than late passage VSMCs (FIG. 8B). The in vitro data was faithfully replicated in vivo, as evidenced by the uptake of EVs-VSMCs^{N1ICD} in aortic VSMCs from aged mice 12 hours post tail vein injection (FIG. 9).

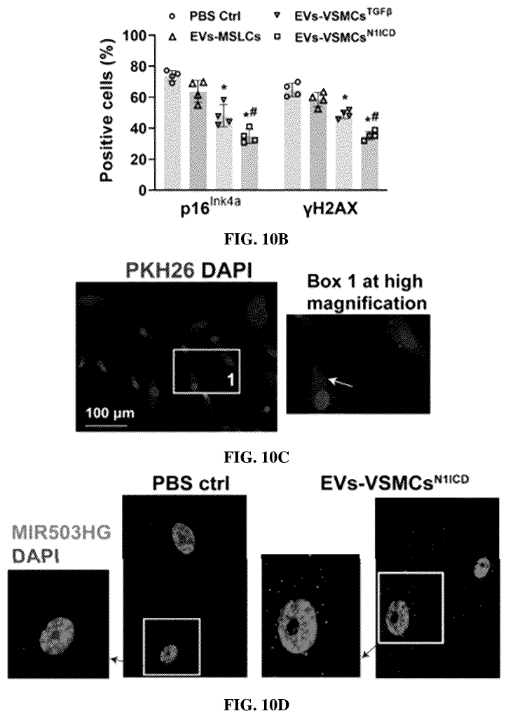

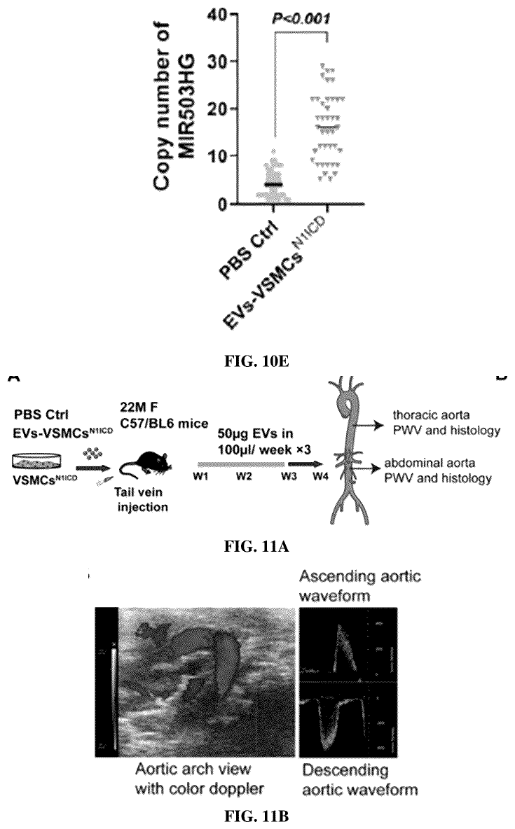

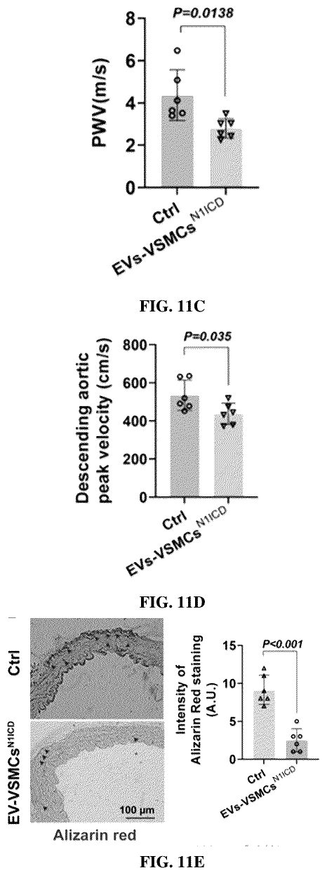

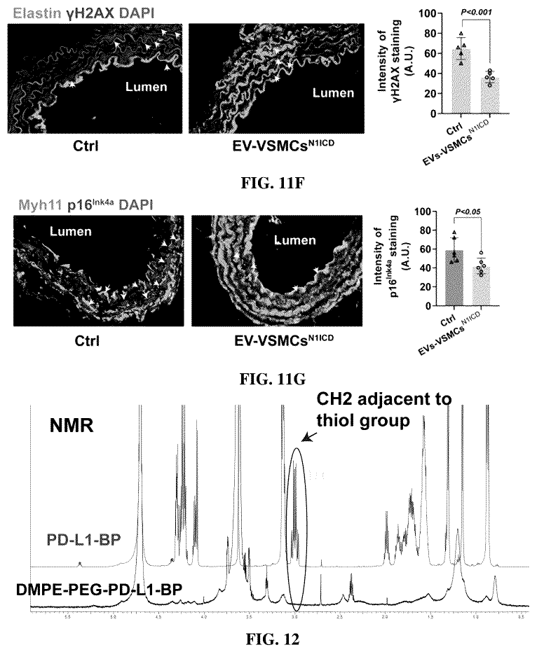

Importantly, EVs released from VSMCs which were differentiated by either TGFβ or Notch1 activation reduced cellular senescence in aged female HAoSMC. This was shown by a decrease in the number of p16^{INK4a} and γH2AX positive cells, compared to those treated with PBS or EVs from MSLCs (FIG. 10A-10B). Interestingly, EVs from more mature VSMCs, VSMCs^{NH1ICD}, exhibited a stronger anti-senescence effect than those from VSMCs^{TGFβ}. An internalization experiment confirmed that EVs derived from VSMCs^{N1ICD} were taken up by replicative senescent female HAoSMC (FIG. 10C). Furthermore, treatment with these EVs increased the copy number of MIR503HG, as detected by the RNAscope ISH assay in these cells, compared to cells treated with PBS (FIG. 10D). The systemic injection of EVs from mature VSMCs into aged female mice exhibited promising therapeutic effects (FIG. 11A), which was indicated by the improvement in aortic stiffness, as evidenced by the lower thoracic pulse wave velocity (PWV) and descending aortic peak velocity (FIG. 11B-11D), reduced calcification (FIG. 11E), and a decrease in the number of senescent cells in the aorta (FIG. 11F-11G), compared to PBS treatment. Based on these promising preliminary findings, it was hypothesize that EVs from iPSC derived mature VSMCs, which are rich in MIR503HG, could alleviate cellular senescence in VSMCs, thereby enhancing the vascular function in aged females. These anti-senescence effects are likely mediated by the targeted delivery of MIR503HG into VSMCs via EVs. Exogenous loading MIR503HG transcript could enhance the therapeutic efficacy of these EVs.

Experimental design and data to be obtained: Isolation and characterization of EVs: EVs will be purified from the medium from the following cells differentiated from hiPSCs (FIG. 4A): 1) MSLCs; 2) VSMCs N1ICD; 3) VSMCs TGF-β. EVs will be isolated using a qEV size exclusion column and subsequently examined in the TEM as outlined in the previous study (Xuan W, et al., Front Cell Dev Biol. 2020:8:11 (“Xuan W. et al.”). The expression of tetraspanins, specifically CD9, CD63, and CD81, will be determined via Western blot analysis Additionally, the protein concentration of the EVs will be quantified using a BCA assay. For the proposed studies, EVs derived from the culture medium of cells at passages 2-4 from various groups will be utilize. For quality control, the composition of miRNAs or lncRNAs in the EVs of mature VSMCs will be validated using RT-PCR (Xuan W, et al.).

Determine whether mature VSMCs derived EVs could attenuate cellular senescence in aged female VSMCs through the delivery of MIR503HG: Due to the fact that multiple bioactive factors are present in the EVs-VSMCs^{N1ICD}, these EVs may also be of therapeutic value to aged males. Therefore, male cells and mice besides female ones will also be include in the in vitro and in vivo study.

In vitro experiments: EVs from different groups as mentioned above will be isolated and purified. Subsequently, the replicative senescent HAoSMC (P10) will be subjected to the following treatments for 72 h: 1) PBS control; 2) EVs-MSLCs; 3) EVs-VSMCs^{TGF-β}; 4) EVs-VSMCs^{N1ICD}; 5) EVs-VSMCs^{N1ICD+MIR503HG silencing} (MIR503HG knockdown by GapmeR). Cellular senescence will be analyzed as described in section (I).

In vivo experiments: 22 months old mice will be randomly divided into the following groups: 1) PBS; 2) EVs-MSLCs; 3) EVs-VSMCs^{TGF-β}; 4) EVs-VSMCs^{N1ICD} 5) EVs-VSMCs^{N1ICD+MIR503HG silencing} (MIR503HG knockdown by GapmeR).

EVs biodistribution analysis: In vivo and ex-vivo EVs biodistribution analysis following different EVs treatments at various doses (50-100 μg) and different time points (6 h, 12 h and 24 h) will be determined as described in the methods section.

Therapeutic effects of EVs: 22 months old mice will receive either PBS or 50 μg of EVs via tail vein injections for 3 times at weekly intervals (FIG. 11A). Four weeks post the initial injection, the PWV will be measure, as outlined in the study (FIG. 11B). The degree of senescence using β-gal staining on the entire mouse aorta will be assess. Immunostaining will be performed for senescence markers (p16^{Ink4a} and γH2AX) and the VSMCs marker α-smooth muscle actin (α-SMA). The quantitative assessment of VSMCs senescence will be determined by counting cells that are double positive for either p16^{Ink4a} or γH2AX and α-SMA. Vascular remodeling will be assessed by quantifying the fluorescence intensity after immunostaining for elastin and collagen. Aortic calcification will be evaluated using Alizarin Red staining. Expression of SASP factors or pro-inflammatory factors (IL-6, IL-8, TNFα) in the aorta following different treatments will be measured.

Determine the therapeutic potential of synthetic MIR503HG transcript using EVs as a delivery vehicle: Studies have demonstrated the effectiveness of synthetic lncRNA therapies in treating diseases in animal models (Mercer T R, et al., Trends Pharmacol Sci. 2022; 43:269-280). In addition, EVs from mature VSMCs with higher level of MIR503HG and targeted VSMC property exhibited superior anti-senescent effects on aged female VSMCs compared to other EVs in the pilot study (FIG. 10A-10E). Therefore, augmenting the enrichment of MIR503HG in these EVs via the direct loading of synthesized MIR503HG transcripts, could enhance the therapeutic efficacy of the EVs. In order to test this hypothesis, 22 months old mice will receive either EVs-VSMCs^{N1ICD} or EVs-VSMCs^{N1ICD} loaded with synthesized MIR503HG via tail vein injection (50 μg) for 3 times at weekly intervals. Four weeks post initial injection, the therapeutic effects will be evaluated through PWV measurement and cellular senescence assessments as described above.

### Methods IIA:

Generation of iPS cells derived mature VSMCs: Human iPSCs will be treated with SB431542 for 10 days to induce MSC-like cells differentiation followed by treatment with TGF-β or transient N1ICD overexpression using adenovirus vector for converting them into VSMCs (FIG. 4A).

EVs isolation: EVs will be isolated as described by us using size exclusion column (SEC) method (Xuan W, et al., Stem Cell Res Ther. 2021; 12:131).

EVs internalization and tracking: PalmGRET reporter will be used for EVs labelling for in vivo tracking and biodistribution analysis as described (Wu A Y, et al., Adv Sci (Weinh). 2020:7:2001467). Briefly, the pLenti-PalmGRET plasmid (Addgene) is used to generate stable PalmGRET-expressing iPSCs with transfection of pLenti-PalmGRET lentivirus vector and puromycin selection. These cells will be then differentiated into VSMCs (FIG. 4A), and their EVs can be detected by either GFP or bioluminescence signal. For EVs biodistribution analysis, different organ tissues (heart, liver, spleen, lung, kidney, aorta) from EVs treated mice are homogenized, lysed, and centrifuged. The bioluminescence signal in the supernatants will be measured. The accumulation of EVs in VSMCs will be determined by double-positive GFP and VSMCs marker (α-SMA, Myh11, elastin) staining in the aorta. Aortic MIR503HG expression will be analyzed after EVs intravenous injection. For in vivo and ex vivo organs imaging, bioluminescence signal will be captured using a Xenogen IVIS imaging system.

Synthesis of MIR503HG Transcripts and Loading into EVs: The synthesis of the MIR503HG transcript (ENST00000414769.2) will be carried out by Creative Biolabs (USA), utilizing the in vitro transcription method previously described (Feng Y, et al., Methods Mol Biol. 2014; 1165:115-143). The MIR503HG transcript will be encapsulated into the EVs via electroporation in 1:1 ratio (Wu X, et al., Eur Heart J. 2023; 44:1761-1763).

Measurement of pulse wave velocity (PWV): Aortic stiffness in mice is measured by the pulse-wave velocity (PWV) technique using Vevo 3100 system (Visual sonics) as described (Tang M, et al., Am J Transl Res. 2021; 13:1352-1364). PWV of thoracic and abdominal aortas will be measured using the Vevo LAB software.

Expected results: In vitro experiments, it is expected that EVs-VSMCs^{N1ICD} will reduce cellular senescence of aged female HAoSMC by decreasing the expression of p21, γH2Ax, p16^{Ink4a} and SASP. Deletion of MIR503HG in EVs-VSMCs^{N1ICD} will diminish the protective effects of EVs. This data will support the rationale that MIR503HG mediates the anti-senescence effects of EVs-VSMCs^{N1ICD} in aged female cells. In vivo experiments, based on preliminary study on the preferential uptake of EVs-VSMCs^{N1ICD} (FIG. 8A-8B) it is expected higher luciferase signal from the homogenized aorta tissue in a dose-dependent manner from the mice treated with EVs-VSMCs^{N1ICD} compared to mice treated with other EVs. It is also predict that EVs from VSMCs^{N1ICD} will effectively reduce accumulation of senescent cells, SASP-related factors, the collagen deposition, and aortic calcification in the aorta resulting in improvement of PWV in these mice compared with EVs from non-VSMCs^{N1ICD}. Deletion of MIR503HG from EVs-VSMCs^{N1ICD} will abrogate these anti-senescence effects.

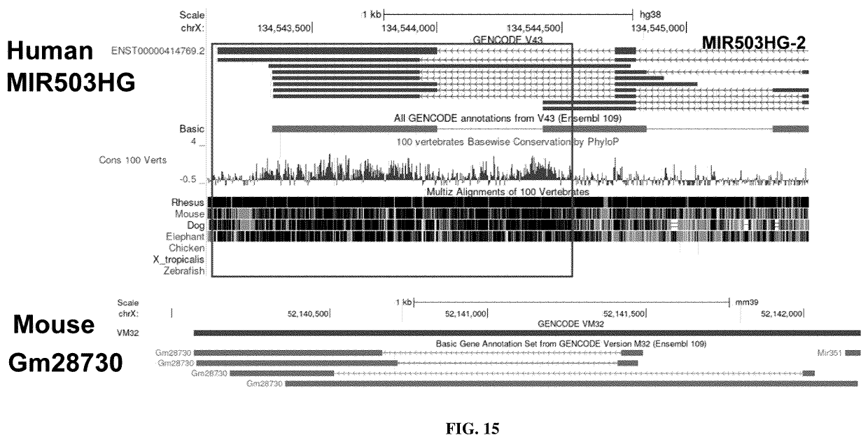

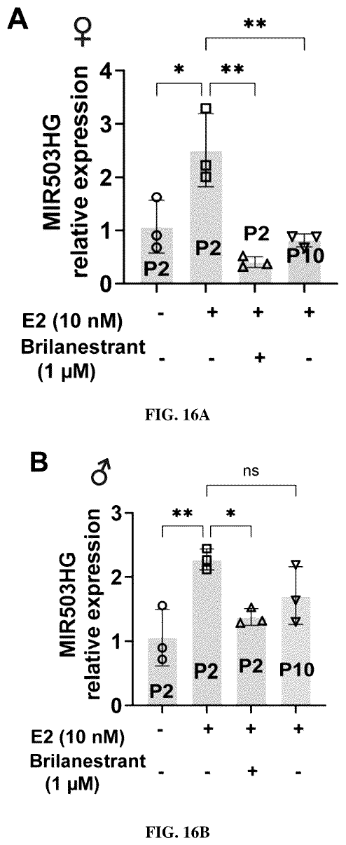

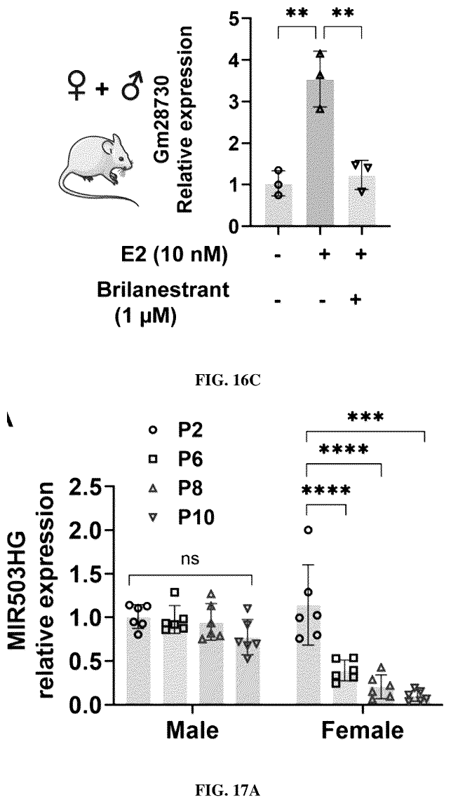

Due to the importance of EVs for future translational applications, EVs from human cells were developed. However, potential limitation of using human iPSCs-derived cells is the possibility that the human MIR503HG transcript from EVs may not effectively target murine cells. However, the downstream region of the MIR503HG locus is conserved (FIG. 15),
and its secondary structure, which reflects the activity of the lncRNA, is also highly conserved (Muys B R, et al., PLoS One. 2016; 11: e0151560). The preliminary data show that E2 treatment could enhance MIR503HG expression in both mouse and human VSMCs (FIGS. 16A-16C).

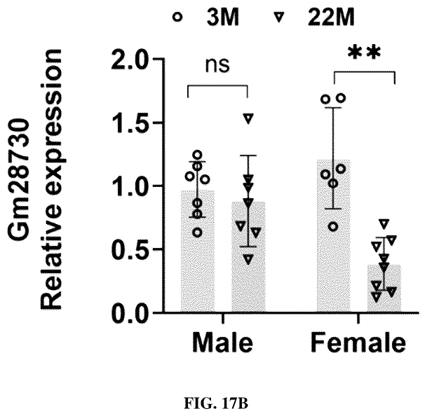

Additionally, the decreased expression of MIR503HG in aged female human and mouse vascular cells was observed (FIGS. 17A-17B), suggesting the functional conservation of MIR503HG. Furthermore, the potential interacting protein, HuR, is a conserved RNA-binding protein (Lopez de Silanes I, et al., Proc Natl Acad Sci USA. 2004; 101:2987-2992; and Majumder M, et al., Adv Drug Deliv Rev. 2022; 188:114442). EVs-VSMCs^{N1ICD} treatment ameliorated vascular senescence in aged female mice (FIG. 11A-11G), therefore human MIR503HG may target murine cells. Consequently, the therapeutic efficacy of these EVs will be further enhanced by loading synthesized human MIR503HG. However, it is possible that there may be no discernible difference in therapeutic efficacy of EVs and EVs loaded with MIR503HG due to low loading efficiency. If this is the case, the loading strategies will be refined, including optimization of electroporation conditions, the mix ratio of EVs and MIR503HG, and EVs-lipid nanoparticles hybridization. MIR503HG and EVs will also be label with fluorescent dyes and nano flow cytometry to monitor the loading efficiency. EVs-VSMCs^{N1ICD} may exhibit protective effects in senescent male VSMCs and aged male mice. However, either silencing or exogenous loading of MIR503HG will not influence the effects of EVs in the male cells and mice. Experimental approaches for animal studies due to the availability of sufficient quantities of EVs. Efficient, and scalable methods for EV production by mass culture of iPSCs will be employed in a 3D bioreactor in the lab and the use of tangential flow filtration for EV purification (Niagara M I, et al., Circ Res. 2007; 100:545-555).

(IIB) Determine whether extracellular vesicles bioengineered with programmed death-ligand 1 binding peptide target senescent VSMC's for increased therapeutic efficacy.

The surface components of EVs are partly responsible for their circulation kinetics and biodistribution (Murphy D E, et al., Exp Mol Med. 2019; 51:1-12 (“Murphy, et al.,”))⁷¹. A previous study reported that intravenously administered EVs could be rapidly cleared from circulation (Murphy, et al.; and de Jong B, et al., Pharmaceutics. 2020; 12, doi: 10.3390/pharmaceutics12111006). EVs from VSMCs^{N1ICD} showed preferential internalization by VSMCs in vitro (FIG. 8A-8B) and had strong therapeutic effect on vascular senescence (FIG. 11A-11G). On the other hand, internalization of EVs by senescent VSMCs was impaired as compared to non-senescent VSMCs (FIG. 8A-8B). Therefore, it is necessary to explore approaches to increase their delivery to the senescent VSMCs for therapeutic effects. EVs can be modified with targeting peptide to enhance their migration or homing to senescent VSMCs. PD-L1, the immune checkpoint protein is expressed by a broader range of cells (Grabie N, et al., Circulation. 2007; 116:2062-2071). PD-L1 expression can be induced in VSMCs by inflammatory stimuli (Koga N, et al., Arterioscler Thromb Vasc Biol. 2004; 24:2057-2062). Recent studies reported that PD-L1 expression was upregulated in senescent cells (Onorati A, et al., Mol Cell Biol. 2022; 42: e0017122; and Wang T W, et al., Nature. 2022; 611:358-364). The enhanced PD-L1 expression was observed in p16^{Ink4a} dependent manner during aging and chronic inflammation (Julia Majewska A A, et al., bioRxiv. doi: doi.org/10.1101/2023.01.30.524522). However, the expression pattern of PD-L1 remains to be unknown in VSMCs during aging. PD-L1 expression was significantly upregulated both in replicative senescent HAoSMC (FIG. 6A) and in the aorta from aged mice (FIG. 6B). Therefore, modifying the surface of EVs to incorporate peptides to target PD-L1 could possibly enhance their delivery efficacy to senescent VSMCs. Polyethylene glycol (PEG), a hydrophilic polymer, is known to increase the circulation time of nanoparticles (Suk J S, et al., Adv Drug Deliv Rev. 2016; 99:28-51; and Shi L, et al., Nanoscale. 2021; 13:10748-10764). PEGylation increased the circulation time of EVs and reduced nonspecific interactions with cells (Murphy D E, et al., Exp Mol Med. 2019; 51:1-12). To further explore this possibility, the surface of VSMCs^{N1ICD} derived EVs were modified with PD-L1 binding peptide (PD-L1-BP, CVRARTR (SEQ ID NO: 1)) by conjugating PD-L1-BP with a PEG-conjugated phospholipid derivative (DMPE-PEG) (FIG. 6D) to fabricate PD-L1-BP engineered EVs (PD-L1-BP-EVs) with PD-L1 targeting ability (FIG. 6C). Nuclear magnetic resonance (NMR) result confirmed the successful conjugation of PD-L1-BP and DMPE-PEG (FIG. 6E). cyanine 5.5 (Cy5.5) dye was also conjugated on PD-L1-BP conjugates for visualization. EVs modified with Cy5.5-PD-L1-BP were referred to as Cy5.5-PD-L1-BP-EVs. The conjugate of Cy5.5-DMPE-PEG (Cy5.5-PEG) was used as a control. EVs modified with Cy5.5-PEG were referred to as Cy5.5-PEG-EVs. Using a co-culture system with early and late passages of HAoSMC and HAoEC (FIG. 6F), PD-L1-BP engineered EVs had better internalization with higher Cy5.5 intensity in replicative senescent HAoSMC compared with Cy5.5-PEG-EVs (FIGS. 6G-6H). Furthermore, PD-L1-BP-engineered EVs also exhibited higher uptake (higher Cy5.5 intensity) by the aorta from old mice after 3 h of systemic delivery (FIGS. 6I-6J). These preliminary results indicate that engineering EVs with PD-L1-BP could improve their targeting efficiency to senescent VSMCs. Taken together, it is hypothesize that enhanced targeting of senescent VSMCs by EVs through modifying their surfaces using PD-L1 binding peptide.

### Experimental Design:

Fabrication and characterization of PD-L1-BP engineered EVs: First, the surface of EVs from VSMCs^{N1ICD} will be modified with a PD-L1-binding peptide by hydrophobic insertion, per the pilot study (FIG. 15B). Next, the modified EVs as described in section IIA will be characterized. For potential future application for using these engineered EVs as a delivery system targeting senescent VSMCs, male and female mice will be included in biodistribution, targeting capabilities and toxicity experiments.

EVs biodistribution analysis: EVs isolated from pLenti-PalmGRET expressing cells will be used for PD-L1-BP engineering. The biodistribution study will be performed as described in section (IIA).

Determine whether PD-L1-BP engineering enhances the targeting capabilities of EVs towards senescent VSMCs in vivo: To investigate whether PD-L1-BP engineering enhances the EVs targeting to senescent VSMCs in vivo, 22 months old p16-3MR mice (senescent cell reporter mice with RFP) will be injected with 50 μg of either Cy5.5-PEG-EVs or Cy5.5-PD-L1-BP-EVs via tail vein. The aorta will be harvested 3 hours post-injection. VSMCs will be identified by α-SMA staining and the targeting efficiency will be evaluated by the number of Cy5.5 positive senescent VSMCs (α-SMA and RFP) in the aorta sections.

Determine whether PD-L1-BP engineered EVs enhance therapeutic effects on vascular senescence: 22 months old female mice will be randomly divided into the following groups: 1) EVs-VSMCs^{N1ICD}; 2) PD-L1-BP-EVs-VSMCs^{N1ICD} Each group will receive 50 μg of EVs via tail vein injection for 3 times at weekly intervals. Four weeks after the first injection, PWV, vascular senescence, and aortic calcification will be analyzed as described in section IIA. Mice with PBS treatment will serve as control.

Systematic toxicity evaluation: 22 months old mice will be injected with PD-L1-BP-EVs-VSMCs^{N1ICD} (50 μg per mouse), or PBS. At 1 week and 4 weeks post injection, serum samples will be collected for alanine transaminase and aspartate aminotransferase tests to assess liver toxicity. In addition, the key organs including heart, lung, liver, and spleen will be harvested for H&E staining to assess pathological changes.

### Methods for IIB:

Engineering EVs with PD-L1 binding peptide (PD-L1-BP): PD-L1-BP [Cys-Val-Arg-Ala-Arg-Thr-Arg (CVRARTR) (SEQ ID NO: 1] (Moon Y, et al., Theranostics. 2022; 12:1999-2014) synthesized by GenScript is covalently bound to DMPE-PEG5000-maleimide to synthesize DMPE-PEG-PD-L1-BP, which could be inserted into the membrane of EVs. EVs are incubated with 5-10 μM DMPE-PEG-PD-L1-BP at room temperature for 30 min to fabricate EVs-PD-L1-BP.

Expected results: it is anticipated that the modification of EVs will not substantially alter their morphology, size distribution, or cargo contents. Increased uptake of PD-L1-BP-modified EVs by aortic senescent VSMCs is predicted. This would be demonstrated by an enhanced luciferase signal and an increase in the number of Cy5.5 positive senescent VSMCs in the aorta, compared to treatment with non-modified EVs. EVs engineered to carry PD-L1-BP will improve therapeutic efficacy due to their enhanced targeting of senescent VSMCs. It is possible that the use of polyethylene glycol tagging peptide to EVs may cause toxicity to body organs and no significant toxicity in the key organs is anticipated, since PEG is an FDA-approved polymer 81 and well tested and widely used in clinical procedures. 50 μg of EVs is proposed for injection based on the pilot study. It is possible that aging mice may be sensitive to the amount of PEG leading to toxicity. If this is the case, the dosage of EVs will be adjusted for each injection and the injection frequency as well.

Rigor and reproducibility: statistically powered sample sizes (calculated according to values for power=0.80 and significance level=0.05). All data values will be expressed as mean±SE and datasets will be assessed for suitability for parametric tests. Comparison between 2 mean values will be evaluated by an unpaired Student 2-tailed t-test, and between 3 or more groups will be evaluated by 1-way ANOVA followed by Bonferroni post-hoc analysis. Considerations for inter-operator variability, blind measurements by researchers to ensure data reproducibility and robustness will be given. Robust experimental design describing methods, inclusion and exclusion criteria and randomization will be adopted. To ensure that the experiments are rigorously designed, at least three independent replicates will be performed.

Sex as a Biological Variable: HAoSMC from both sexes will be utilized to investigate the sex dependent differences affecting estrogen/ERα-MIR503HG signaling pathway during replicative senescence. Aged mice from both sexes will be utilized to evaluate the sex differences in the therapeutic effectiveness of EVs on vascular senescence.

Mature VSMCs from induced pluripotent stem cells (iPSCs) can be generated using a combination of the small molecule SB43152 and transient Notch1 activation for higher expression of contractile markers. EVs derived from these mature VSMCs are abundant in MIR503HG with a homing-target capability towards VSMCs. The anti-senescent effects of these EVs on VSMCs can be evaluated using an in vitro model of replicative senescence and naturally aged female mice. It can also be investigated whether the mechanistic effect of EVs against VSMCs senescence is partially attributable to the delivery of MIR503HG by the EVs. The rationale is further supported by the findings that PD-L1 expression is increased in replicative senescent VSMCs and in aged aorta. Consequently, it can be explore whether engineering the surfaces of these EVs with a PD-L1 binding peptide could enhance targeted delivery to senescent VSMCs, thereby amplifying their rejuvenating effect on vascular aging. In addition, the therapeutic value of these EVs can also be determined in the aged male mice.

The study can uncover a molecular mechanisms of vascular aging and provide a therapeutic strategy for reducing vascular senescence in aged/postmenopausal women.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

What is claimed is:

1. An extracellular vesicle comprising a targeting peptide and an active agent,
wherein the targeting peptide is a programmed death-ligand 1 (PD-L1) binding peptide having at least 80% sequence identity to SEQ ID NO: 1, and
wherein the targeting peptide targets senescent cells.

2. The extracellular vesicle of claim 1, wherein the targeting peptide is conjugated to a PEG-conjugated phospholipid derivative.

3. The extracellular vesicle of claim 2, wherein the PEG-conjugated phospholipid derivative is DMPE-PEG-maleimide.

4. The extracellular vesicle of claim 1, wherein the targeting peptide conjugated to a PEG-conjugated phospholipid derivative is DMPE-PEG-PD-L1-BP.

5. The extracellular vesicle of claim 1, wherein the extracellular vesicle is an extracellular vesicle derived from smooth muscle cells.

6. The extracellular vesicle of claim 5, wherein the smooth muscle cells are vascular smooth muscle cells (VSMCs)).

7. The extracellular vesicle of claim 1, wherein the extracellular vesicle is an extracellular vesicle derived from vascular smooth muscle cells (VSMCs) from human induced pluripotent stem cells (hiPSC).

8. The extracellular vesicle of claim 1, wherein the active agent is anti-senescence active agent.

9. The extracellular vesicle of claim 8, wherein the anti-senescence active agent is a nucleic acid, a protein/peptide, a small molecule, or any combination thereof.

10. The extracellular vesicle of claim 9, wherein the anti-senescence active agent is a nucleic acid.

11. The extracellular vesicle of claim 10, wherein the nucleic acid comprises double stranded DNA, single-stranded DNA, complexed DNA, encapsulated DNA, naked RNA, encapsulated RNA, messenger RNA (mRNA), tRNA, short interfering RNA (siRNA), double stranded RNA (dsRNA), micro-RNA (miRNA), a long non-coding RNAs (lncRNAs), antisense RNA (asRNA), self-amplify mRNA (saRNA), guide RNA (gRNA), CRNA, or any combination thereof.

12. The extracellular vesicle of claim 11, wherein the nucleic acid is a long non-coding RNAs.

13. The extracellular vesicle of claim 12, wherein the long non-coding RNAs (lncRNAs) is a MIR503HG long non-coding RNAs (lncRNAs) having at least 80% sequence identity to SEQ ID NO: 2, MIR143HG long non-coding RNAs (lncRNAs) having at least 80% sequence identity to SEQ ID NOs: 3 or 4, or any combination thereof.

14. A pharmaceutical composition comprising a pharmaceutically acceptable carrier and an effective amount of the extracellular vesicle of claim 1.

15. A method for delivering an active agent into a senescent cell, comprising:
introducing into the senescent cell an extracellular vesicle of claim 1.

16. A method for treating or preventing conditions, diseases, or disorders related to, associated with, or caused by cellular senescence in a subject in need thereof, the method comprising administering to a subject in need thereof an extracellular vesicle of claim 1.

17. A method of treating, preventing, or delaying onset of a senescent cell-associated disease or disorder in a subject who has a senescent cell-associated disease or disorder or who has at least one predisposing factor for developing the senescent cell-associated disease or disorder, the method comprising administering to a subject in need thereof an extracellular vesicle of claim 1.

18. A method of treating or preventing vascular diseases, the method comprising administering to a subject in need thereof an extracellular vesicle of claim 1.

19. A method for reducing cellular senescence in human aortic smooth muscle cells (HAoSMC) by decreasing the expression of senescence cell associated molecules, the method comprising administering to a subject in need thereof an extracellular vesicle of claim 1.

20. A method for reducing cellular senescence by restoring expression levels of MIR503HG in senescent smooth muscle cells, the method comprising administering to a subject in need thereof an extracellular vesicle comprising a therapeutically effective amount of a MIR503HG long non-coding RNAs (lncRNAs) having at least 80% sequence identity to SEQ ID NO: 2, and a targeting peptide, wherein the targeting peptide targets senescent cells.
